(19) United States
(12) Patent Application Publication
(10) Pub. No.: US 2024/0000782 A1
(43) Pub. Date: Jan. 4, 2024

(54) METHODS AND COMPOSITIONS FOR TREATING AN OPHTHALMIC CONDITION

(71) Applicant: Telios Pharma, Inc., Redwood City, CA (US)

(72) Inventors: Wayne Philip Rothbaum, Delray Beach, FL (US); Michael Weiser, New York, NY (US)

(73) Assignee: Telios Pharma, Inc., Redwood City, CA (US)

(21) Appl. No.: 18/039,673

(22) PCT Filed: Dec. 2, 2021
(86) PCT No.: PCT/US2021/061555

§ 371 Date: May 31, 2023

Related U.S. Application Data
(60) Provisional application No. 63/120,510, filed on Dec. 2, 2020.

(51) Int. Cl. A61K 31/506

(52) U.S. Cl. CPC A61K 31/506

(57) ABSTRACT

Therapeutic methods and pharmaceutical compositions for treating an ophthalmic condition including dry eye syndrome and uveitis in a human subject are described. In certain embodiments, the disclosure includes therapeutic methods using a BTK inhibitor.

### FIELD OF THE DISCLOSURE

Methods and compositions of treating an ophthalmic condition including dry eye syndrome and uveitis using a Bruton's Tyrosine Kinase (BTK) inhibitor are disclosed herein.

### BACKGROUND

Dry eye disease (also referred to as kertoconjunctivitis sicca or dry eye syndrome) is a multifactorial disorder characterized by either a decreased tear production or an increased tear film evaporation. Patients that suffer from dry eye disease have symptoms including eye irritation, redness, ocular discharge, and decrease in tear volume. Dry eye disease results in a loss of integrity of the tear film, which leads to ocular surface inflammation. Nonpharmaceutical treatments for dry eye disease include artificial tears, punctal plugs, and autologous serum drops. FDA-approved pharmaceutical treatments for dry eye disease include immunomodulators and immunosuppressive agents such as cyclosporin and corticosteroids. Patients with severe dry eye disease frequently have a poor response to normal twice per day dosing and often benefit from an increased dosing frequency. However, with increased application requirements, the rate of patient compliance typically decreases.

Uveitis is an inflammation of the middle layer of the eye (urea). Patients suffering from uveitis have symptoms including redness, pain, light sensitivity, blurred vision, and dark floating spots in the field of vision. Uveitis can lead to permanent vision loss. Possible causes of uveitis are infection, injury, or an autoimmune or inflammatory disease. In some cases, the cause of uveitis is not clear. Common treatments for uveitis include eye drops that reduce the inflammation. Uveitis that is the result of an infection may be treated with antibiotics or antiviral agents.

Bruton's tyrosine kinase (BTK) is a Tec family non-receptor protein kinase, expressed in B cells and myeloid cells. Functional mutations in BTK in humans results in the primary immunodeficiency disease called XLA which is characterized by a defect in B cell development with a block between pro- and pre-B cell stage. This results in an almost complete absence of B lymphocytes in humans causing a pronounced reduction of serum immunoglobulin of all classes.

The present disclosure relates to methods of treating an ophthalmic condition in a human subject with a BTK inhibitor.

### SUMMARY

In one aspect, the present disclosure relates to method of treating an ophthalmic condition in a human subject in need thereof comprising: administering to the human subject an amount of a Bruton's Tyrosine Kinase (BTK) inhibitor compound effective to treat the ophthalmic condition in the human subject.

In one aspect, the present disclosure relates to a method of reducing an immune response in a human subject having an ophthalmic condition, comprising administering to the human subject an amount of a Bruton's Tyrosine Kinase (BTK) inhibitor compound effective to reduce an immune response in the human subject.

In an embodiment, the BTK inhibitor compound is 1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof.

In an embodiment, administering the BTK inhibitor compound reduces inflammation in an eye of the human subject.

In an embodiment, the ophthalmic condition is ocular inflammation.

In an embodiment, the ophthalmic condition is selected from dry eye disease, uveitis, post-operative ocular inflammation, corneal transplantation, ocular graft-versus-host disease (GVHD), allergy, allergic conjunctivitis, non-allergic conjunctivitis, or infectious conjunctivitis.

In an embodiment, the ophthalmic condition is dry eye disease.

In an embodiment, the ophthalmic condition is aqueous-deficient dry eye disease.

In an embodiment, the ophthalmic condition is hyperevaporative dry eye disease.

In an embodiment, the ophthalmic condition is mixed aqueous-deficient and hyperevaporative dry eye disease.

In an embodiment, the ophthalmic condition is uveitis.

In an embodiment, the ophthalmic condition is infectious uveitis.

In an embodiment, the ophthalmic condition is non-infectious uveitis.

In an embodiment, the ophthalmic condition is anterior uveitis.

In another aspect, the ophthalmic condition is intermediate uveitis.

In an embodiment, the ophthalmic condition is posterior uveitis.

In an embodiment, the ophthalmic condition is panuveitis.

In an embodiment, administering comprises topical administration to an eye of the human subject.

In an embodiment, administering comprises intraocular injection to an eye of the human subject.

In an embodiment, administering comprises intravitreal injection to an eye of the human subject.

In an embodiment, administering comprises periocular administration to the human subject.

In an embodiment, administering comprises oral administration to the human subject.

In an embodiment, administering comprises intravenous injection (including intravenous infusion) to the human subject.

In an embodiment, the compound is administered as nanoparticles comprising the compound.

In an embodiment, the compound is in a dosage form selected from a solution, suspension, emulsion, microemulsion, ointment, gel, hydrogel, drug delivery device, tablet, or capsule.

In an embodiment, the drug delivery device is an ocular insert for sustained release of the BTK inhibitor compound.

In an embodiment, the dosage form is a sustained release form, an extended release form, a controlled release form, or a combination thereof.

In an embodiment, the sustained release, extended release, or controlled release dosage form comprises a pegylated BTK inhibitor.

In an embodiment, the compound is administered as particles that self-aggregate into a depot upon administration.

In an embodiment, the particles further comprise a polymer.

In an embodiment, the polymer is selected from the group consisting of chitosan, gelatin, sodium alginate, albumin, poly-L-lactide (PLLA), poly(lactic acid) (PLA), poly(glycolic acid)(PGA), poly(lactic co-glycolic acid) (PLGA), polycaprolactone, poly(lactide co-caprolactone), poly(methyl methacrylates), poloxamer, poly(ethylene glycol) (PEG), PEG-PLLA, PEG-PLGA, poly(methyl vinyl ether/maleic anhydride), cellulose acetate phthalate, and combinations thereof.

In an embodiment, the polymer is poly(lactic co-glycolic acid) (PLGA), PEG-PLGA, or a combination thereof.

In an embodiment, T-cells in an eye of the human subject overexpress lymphocyte function-associated antigen (LFA-1).

In an embodiment, administering the compound decreases the expression of LFA-1.

In an embodiment, the compound inhibits intercellular adhesion molecule 1 (ICAM-1) in an eye of the human subject.

In an embodiment, the ICAM-1 is present on antigen-presenting cells (APCs) in the eye of the human subject.

In an embodiment, the ICAM-1 is present on vascular endothelial cells in the eye of the human subject.

In an embodiment, the ICAM-1 is present on corneal epithelial cells in the eye of the human subject.

In an embodiment, administering the compound reduces levels of inflammatory cytokines.

In an embodiment, the inflammatory cytokines are selected from IL-1β, IL-6, INF-γ, TNF-α, or a combination thereof.

In an embodiment, administering the compound reduces ocular surface APCs, maturation of APCs, or both.

In an embodiment, the APCs are monocytes, macrophages, dendritic cells, B cells, or combinations thereof.

In an embodiment, the human subject has a marker of an ophthalmic condition.

In an embodiment, the marker is elevated inflammatory cytokines, elevated chemokines, elevated matrix metalloproteinases (MMPs), elevated toll-like receptor 2 (TLR2), elevated nuclear factor-kappa B (NF-κB), elevated tumor necrosis factor alpha (TNF-α), or combinations thereof.

In an embodiment, the inflammatory cytokines are selected from IL-1β, IL-6, INF-γ, TNF-α, or a combination thereof.

In an embodiment, the human subject has an auto-immune disease or an inflammatory disease in addition to the ophthalmic condition.

In an embodiment, the auto-immune disease or inflammatory disease is rheumatoid arthritis, Sjögren's syndrome, Vogt-Koyanagi-Harada (VKH) disease, juvenile idiopathic arthritis, Behçet's disease, systemic sarcoidosis, spondyloarthropathy (such as HLA-B27 associated spondyloarthropathy), Blau syndrome, or IgG-4 related disease (IgG4-RD).

In an embodiment, administering occurs at a frequency of three times a daily, twice daily, once daily, every other day, three times a week, twice a week, weekly, every two weeks, twice a month, monthly, every two months, or every three months.

In an embodiment, the immune response is an innate immune response, an adaptive immune response, or both.

### DETAILED DESCRIPTION

While preferred embodiments of the disclosure are shown and described herein, such embodiments are provided by way of example only and are not intended to otherwise limit the scope of the disclosure. Various alternatives to the described embodiments may be employed in practicing the disclosure.

Unless defined otherwise, all technical and scientific terms used herein have the same meaning as is commonly understood by one of skill in the art to which this disclosure belongs.

The term “amount effective to” or “effective amount” or “therapeutically effective amount” refers to that amount of an active pharmaceutical ingredient or combination of active pharmaceutical ingredients as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, and other factors which can readily be determined by one of ordinary skill in the art. The term also applies to a dose that will induce a particular response in target cells, (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” means quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “Polydispersity Index (PDI)” is defined as the square of the ratio of standard deviation (σ) of the particle diameter distribution divided by the mean particle diameter (2a), as illustrated by the formula: PDI=(σ/2a)². PDI is used to estimate the degree of non-uniformity of a size distribution of nanoparticles, and larger PDI values correspond to a larger size distribution in the particle sample. PDI can also indicate particle aggregation along with the consistency and efficiency of particle surface modifications. A sample is considered monodisperse when the PDI value is less than 0.1.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts.

“Pharmaceutically acceptable carrier” or “pharmaceutically acceptable excipient” is intended to include any and all solvents, dispersion media, coatings, antibacterial and antifungal agents, isotonic, and absorption delaying agents. The use of such media and agents for active pharmaceutical ingredients is well known in the art. Except insofar as any conventional media or agent is incompatible with the active pharmaceutical ingredient, its use in the therapeutic compositions of the disclosure is contemplated. Supplementary active ingredients can also be incorporated into the described compositions.

“Solvate” refers to a compound in physical association with one or more molecules of a pharmaceutically acceptable solvent.

Compounds of the disclosure also include crystalline and amorphous forms of the compounds listed in Table 1, including, for example, polymorphs, pseudopolymorphs, solvates, hydrates, unsolvated polymorphs (including anhydrates), conformational polymorphs, and amorphous forms of the compounds, as well as mixtures thereof.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

Methods of Treating Ophthalmic Conditions and Inflammation with BTK Inhibitors

The present disclosure relates to a method of treating an ophthalmic condition comprising the step of administering to a human in need thereof a Bruton's Tyrosine Kinase (BTK) inhibitor compound. The ophthalmic condition includes ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The BTK inhibitor compound is administered in an amount effective to treat the ophthalmic condition in the human.

The present disclosure also relates to a method of reducing inflammation in an eye of a human in need thereof comprising the step of administering to the human a BTK inhibitor compound. The BTK inhibitor compound is administered in an amount effective to reduce inflammation in the eye of the human.

The present disclosure further relates to a method of reducing an immune response in a human in need thereof having an ophthalmic condition comprising the step of administering to the human a BTK inhibitor compound. The immune response includes an innate immune response, an adaptive immune response, or both. The ophthalmic condition includes ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The BTK inhibitor compound is administered in an amount effective to reduce the immune response in the human having an ophthalmic condition.

The present disclosure also relates to a method of treating an ophthalmic condition comprising the step of administering to a human in need thereof a pharmaceutical composition comprising the BTK inhibitor compound. The ophthalmic condition includes ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The pharmaceutical composition is administered in an amount effective to treat the ophthalmic condition in the human.

The present disclosure also relates to a method of reducing inflammation in an eye of a human in need thereof comprising the step of administering to the human a pharmaceutical composition comprising the BTK inhibitor compound. The pharmaceutical composition is administered in an amount effective to reduce inflammation in the eye of the human.

The present disclosure further relates to a method of reducing an immune response in a human in need thereof having an ophthalmic condition comprising the step of administering to the human a pharmaceutical composition comprising the BTK inhibitor compound. The immune response includes an innate immune response, an adaptive immune response, or both. The ophthalmic condition includes ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The pharmaceutical composition is administered in an amount effective to reduce the immune response in the human having an ophthalmic condition.

The present disclosure relates to a method of decreasing the expression of lymphocyte function-associated antigen (LFA-1) in an eye of a human comprising the step of administering to the human a BTK inhibitor compound (including a pharmaceutical composition comprising the BTK inhibitor compound). In an embodiment, the human has an ophthalmic condition as described herein. The BTK inhibitor compound is administered in an amount effective to decrease expression of LFA-1 in an eye of the human. In an embodiment, T-cells in an eye of a human having an ophthalmic condition overexpress LFA-1 and administration of the BTK inhibitor compound decreases expression of LFA-1 in the eye of the human.

The present disclosure relates to a method of inhibiting intercellular adhesion molecule 1 (ICAM-1) in an eye of the human comprising the step of administering to the human a BTK inhibitor compound (including a pharmaceutical composition comprising the BTK inhibitor compound). In an embodiment, the human has an ophthalmic condition as described herein. The BTK inhibitor compound is administered in an amount effective to inhibit ICAM-1 in an eye of the human. In an embodiment, ICAM-1 is present on antigen-presenting cells (APCs) in the eye of the human, and administration of the BTK inhibitor compound inhibits ICAM-1 on APCs in the eye of the human. In an embodiment, ICAM-1 is present on vascular endothelial cells in the eye of the human, and administration of the BTK inhibitor compound inhibits ICAM-1 on vascular endothelial cells in the eye of the human. In an embodiment, ICAM-1 is present on corneal endothelial cells in the eye of the human, and administration of the BTK inhibitor compound inhibits ICAM-1 on corneal endothelial cells in the eye of the human.

The present disclosure relates to a method of reducing levels of inflammatory cytokines in a human (including in an eye of the human) comprising the step of administering to the human a BTK inhibitor compound (including a pharmaceutical composition comprising the BTK inhibitor compound). In an embodiment, the human has an ophthalmic condition as described herein. The BTK inhibitor compound is administered in an amount effective to reduces levels of inflammatory cytokines in the human (including in the eye of the human). In an embodiment, the inflammatory cytokines are selected from IL-1β, IL-6, INF-γ, TNF-α, or a combination thereof.

The present disclosure relates to a method of reducing ocular surface APCs, maturation of APCs, or both in an eye of a human comprising the step of administering to the human a BTK inhibitor compound (including a pharmaceutical composition comprising the BTK inhibitor compound). In an embodiment, the human has an ophthalmic condition as described herein. The BTK inhibitor compound is administered in an amount effective to reducing ocular surface APCs, maturation of APCs, or both in an eye of the human. In an embodiment, the APCs are monocytes, macrophages, dendritic cells, B cells, or combinations thereof.

In an embodiment, the human has a marker of an ophthalmic condition. In an embodiment, the marker is elevated inflammatory cytokines, elevated chemokines, elevated matrix metalloproteinases (MMPs), elevated toll-like receptor 2 (TLR2), elevated nuclear factor-kappa B (NF-κB), elevated tumor necrosis factor alpha (TNF-α), or combinations thereof. In an embodiment, the inflammatory cytokines are selected from IL-1β, IL-6, INF-γ, TNF-α, or a combination thereof.

In an embodiment, the human has an auto-immune disease or an inflammatory disease in addition to the ophthalmic condition. In an embodiment, the auto-immune disease or inflammatory disease is rheumatoid arthritis, Sjögren's syndrome, Vogt-Koyanagi-Harada (VKH) disease, juvenile idiopathic arthritis, Behçet's disease, systemic sarcoidosis, spondyloarthropathy (such as HLA-B27 associated spondyloarthropathy), Blau syndrome, or IgG-4 related disease (IgG4-RD).

In an embodiment, the BTK inhibitor compound is selected from the compounds listed in Table 1 or a pharmaceutically acceptable salt thereof:

TABLE 1
BTK Inhibitors

| No. | IUPAC Name | Structure |
|---|---|---|
| 1. | Acalabrutinib ((S)-4-(8-amino-3-(1- (but-2-ynoyl)pyrrolidin-2- yl)imidazo[1,5-a]pyrazin-1-yl)-N- (pyridin-2-yl)benzamide) | 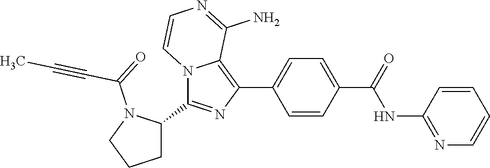 [SMILES: CC#CC(=O)N1CCC[C@H]1c1nc(-c2ccc(C(=O)Nc3ccccn3)cc2)c2c(N)nccn12] |
| 2. | Ibrutinib (1-[(3R)-3-[4-amino-3-(4- phenoxyphenyl)pyrazolo[3,4- d]pyrimidin-1-yl]piperidin-1-yl]prop- 2-en-1-one) | 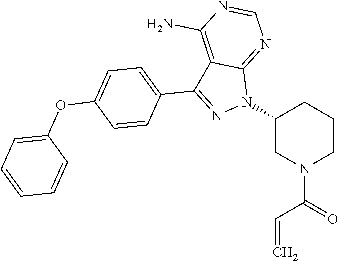 [SMILES: C=CC(=O)N1CCC[C@@H](n2nc(-c3ccc(Oc4ccccc4)cc3)c3c(N)ncnc32)C1] |
| 3. | (7S)-2-(4-phenoxyphenyl)-7-(1-prop- 2-enoylpiperidin-4-yl)-4,5,6,7- tetrahydropyrazolo[1,5-a]pyrimidine- 3-carboxamide | 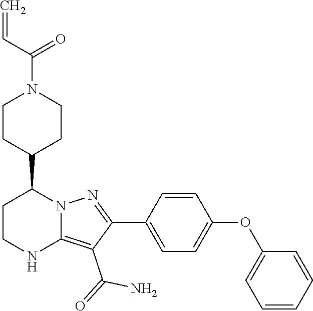 [SMILES: C=CC(=O)N1CCC([C@@H]2CCNc3c(C(N)=O)c(-c4ccc(Oc5ccccc5)cc4)nn32)CC1] |
| 4. | 2-(4-phenoxyphenyl)-7-(1-prop-2- enoylpiperidin-4-yl)-4,5,6,7- tetrahydropyrazolo[1,5-a]pyrimidine- 3-carboxamide | 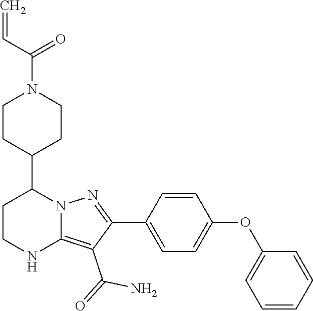 [SMILES: C=CC(=O)N1CCC(C2CCNc3c(C(N)=O)c(-c4ccc(Oc5ccccc5)cc4)nn32)CC1] |
| 5. | (7R)-2-(4-phenoxyphenyl)-7-(1-prop- 2-enoylpiperidin-4-yl)-4,5,6,7- tetrahydropyrazolo[1,5-a]pyrimidine- 3-carboxamide | 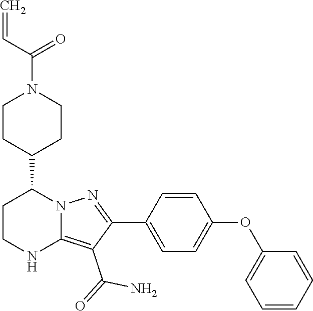 [SMILES: C=CC(=O)N1CCC([C@H]2CCNc3c(C(N)=O)c(-c4ccc(Oc5ccccc5)cc4)nn32)CC1] |
| 6. | 6-amino-9-[(3R)-1-but-2- ynoylpyrrolidin-3-yl]-7-(4- phenoxyphenyl)purin-8-one | 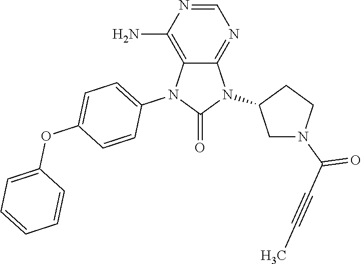 [SMILES: CC#CC(=O)N1CC[C@@H](n2c(=O)n(-c3ccc(Oc4ccccc4)cc3)c3c(N)ncnc32)C1] |
| 7. | N-[3-[[5-fluoro-2-[4-(2- methoxyethoxy)anilino]pyrimidin-4- yl]amino]phenyl]prop-2-enamide | 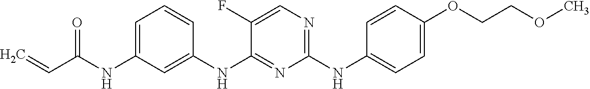 [SMILES: C=CC(=O)Nc1cccc(Nc2nc(Nc3ccc(OCCOC)cc3)ncc2F)c1] |
| 8. | Fenebrutinib (10-[3-(hydroxymethyl)- 4-[1-methyl-5-[[5-[(2S)-2-methyl-4- (oxetan-3-yl)piperazin-1-yl]pyridin-2- yl]amino]-6-oxopyridin-3-yl]pyridin- 2-yl]-4,4-dimethyl-1,10- diazatricyclo[6.4.0.0^{2,6}]dodeca-2(6),7- dien-9-one) | 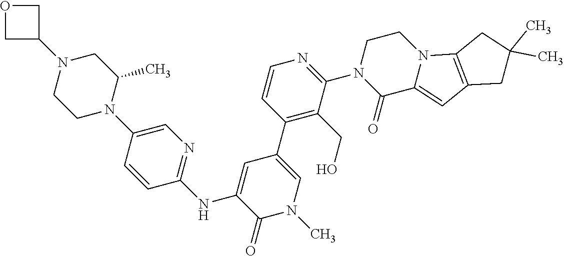 [SMILES: C[C@H]1CN(C2COC2)CCN1c1ccc(Nc2cc(-c3ccnc(N4CCn5c(cc6c5CC(C)(C)C6)C4=O)c3CO)cn(C)c2=O)nc1] |
| 9. | 1-[4-[[[6-amino-5-(4- phenoxyphenyl)pyrimidin-4- yl]amino]methyl]piperidin-1-yl]prop- 2-en-1-one | 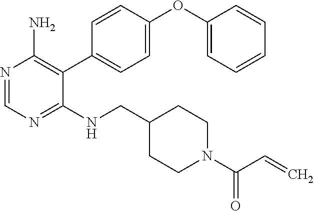 [SMILES: C=CC(=O)N1CCC(CNc2ncnc(N)c2-c2ccc(Oc3ccccc3)cc2)CC1] |
| 10. | 1-[4-[[[6-amino-5-(4- phenoxyphenyl)pyrimidin-4- yl]amino]methyl]piperidin-1-yl]prop- 2-en-1-one | 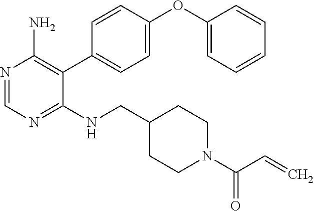 [SMILES: C=CC(=O)N1CCC(CNc2ncnc(N)c2-c2ccc(Oc3ccccc3)cc2)CC1] |
| 11. | (2-chloro-4-phenoxyphenyl)-[4- [[(3R,6S)-6-(hydroxymethyl)oxan-3- yl]amino]-7H-pyrrolo[2,3-d]pyrimi- din-5-yl]methanone | 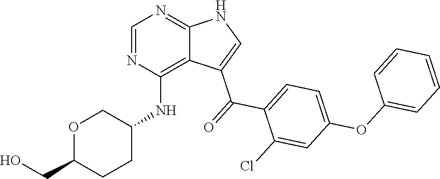 [SMILES: O=C(c1ccc(Oc2ccccc2)cc1Cl)c1c[nH]c2ncnc(N[C@@H]3CC[C@@H](CO)OC3)c12] |
| 12. | N-[3-[6-[4-[(2R)-1,4-dimethyl-3- oxopiperazin-2-yl]anilino]-4-methyl- 5-oxopyrazin-2-yl]-2-methylphenyl]- 4,5,6,7-tetrahydro-1-benzothiophene- 2-carboxamide | 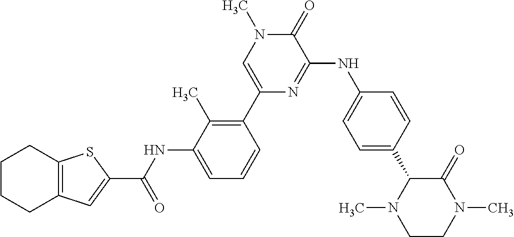 [SMILES: Cc1c(NC(=O)c2cc3c(s2)CCCC3)cccc1-c1cn(C)c(=O)c(Nc2ccc([C@@H]3C(=O)N(C)CCN3C)cc2)n1] |
| 13. | 2-[2-[2-[4-[4-amino-3-(4- phenoxyphenyl)pyrazolo[3,4- d]pyrimidin-1-yl]piperidin-1- yl]ethoxy]ethoxy]-N-[2-(2,6- dioxopiperidin-3-yl)-1,3-dioxo- isoindol-5-yl]acetamide | 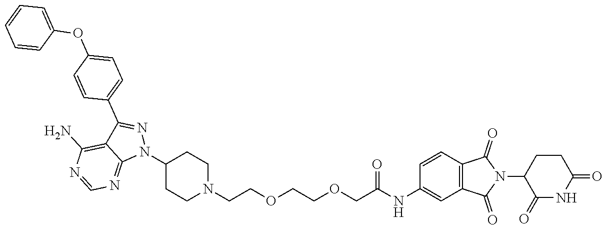 [SMILES: Nc1ncnc2c1c(-c1ccc(Oc3ccccc3)cc1)nn2C1CCN(CCOCCOCC(=O)Nc2ccc3c(c2)C(=O)N(C2CCC(=O)NC2=O)C3=O)CC1] |
| 14. | N-[3-[2-[4-(4-methylpiperazin-1- yl)anilino]furo[3,2-d]pyrimidin-4- yl]oxyphenyl]prop-2-enamide | 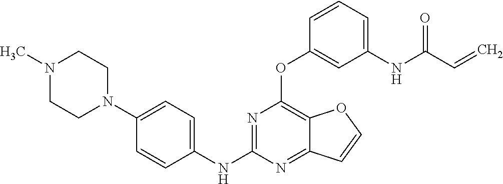 [SMILES: C=CC(=O)Nc1cccc(Oc2nc(Nc3ccc(N4CCN(C)CC4)cc3)nc3ccoc23)c1] |
| 15. | 4-tert-butyl-N-[2-methyl-3-[1-meth- yl-5-[4-(morpholine-4-carbonyl)-3- (prop-2-enoylamino)anilino]-6- oxopyridin-3-yl]phenyl]benzamide | 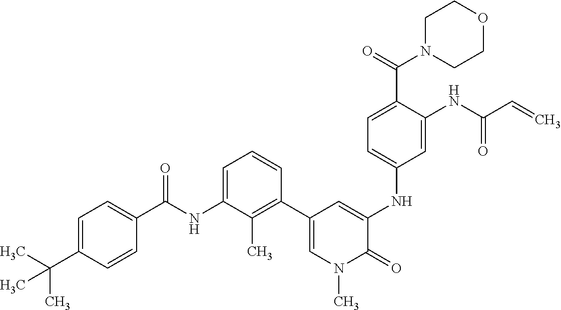 [SMILES: C=CC(=O)Nc1cc(Nc2cc(-c3cccc(NC(=O)c4ccc(C(C)(C)C)cc4)c3C)cn(C)c2=O)ccc1C(=O)N1CCOCC1] |
| 16. | (R,E)-2-(3-(4-amino-3-(2-fluoro-4- phenoxyphenyl)-1H-pyrazolo[3,4- d]pyrimidin-1-yl)piperidine-1- carbonyl)-4-methyl-4-(4-(oxetan-3- yl)piperazin-1-yl)pent-2-enenitrile | 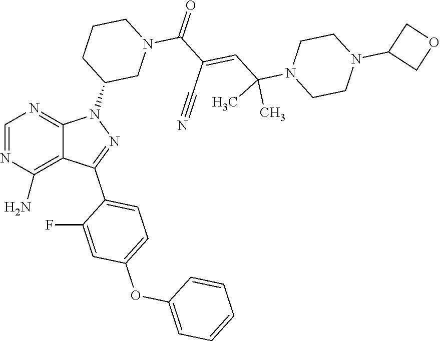 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1] |
| 17. | Branebrutinib ((S)-4-(3-(but-2-yn- amido)piperidin-1-yl)-5-fluoro-2,3- dimethyl-1H-indole-7-carboxamide) | 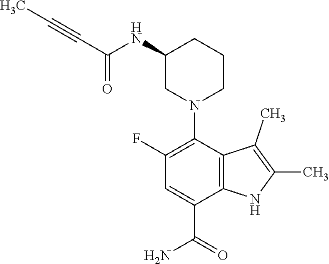 [SMILES: CC#CC(=O)N[C@H]1CCCN(c2c(F)cc(C(N)=O)c3[nH]c(C)c(C)c23)C1] |
| 18. | 4-(tert-Butyl-N-(2-methyl-3-(4- methyl-6-((4-(morpholine-4- carbonyl)phenyl)amino)-5-oxo-4,5- dihydropyrazin-2-yl)phenyl)benz- amide | 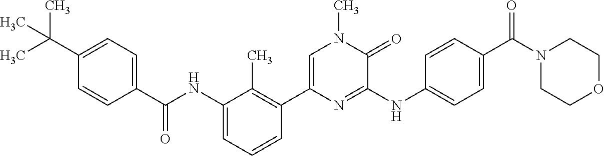 [SMILES: Cc1c(NC(=O)c2ccc(C(C)(C)C)cc2)cccc1-c1cn(C)c(=O)c(Nc2ccc(C(=O)N3CCOCC3)cc2)n1] |
| 19. | N-(1-(7H-Pyrrolo[2,3-d]pyrimidin-4- yl)piperidin-3-yl)-2-((3- chlorophenyl)amino)acetamide | 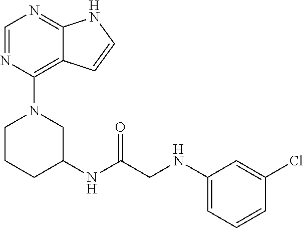 [SMILES: O=C(CNc1cccc(Cl)c1)NC1CCCN(c2ncnc3[nH]ccc23)C1] |
| 20. | 6-cyclopropyl-8-fluoro-2-[2- (hydroxymethyl)-3-[1-methyl-5-[[5- (4-methylpiperazin-1-yl)pyridin-2- yl]amino]-6-oxopyridin-3- yl]phenyl]isoquinolin-1-one | 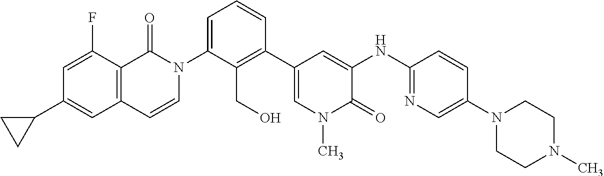 [SMILES: CN1CCN(c2ccc(Nc3cc(-c4cccc(-n5ccc6cc(C7CC7)cc(F)c6c5=O)c4CO)cn(C)c3=O)nc2)CC1] |
| 21. | N-[5-[9-[4- (methanesulfonamido)phenyl]-2- oxobenzo[h]1,6]naphthyridin-1-yl]-2- methylphenyl]prop-2-enamide | 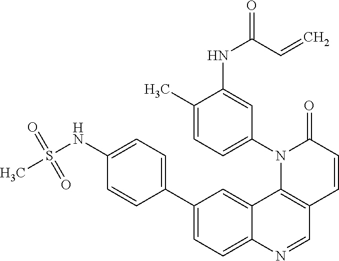 [SMILES: C=CC(=O)Nc1cc(-n2c(=O)ccc3cnc4ccc(-c5ccc(NS(C)(=O)=O)cc5)cc4c32)ccc1C] |
| 22. | 4-(4-((4-((3-acrylamidophenyl)a- mino)-5-fluoropyrimidin-2- yl)amino)phenoxy)-N- methylpicolinamide | 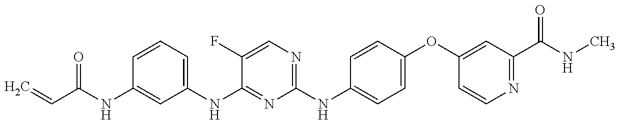 [SMILES: C=CC(=O)Nc1cccc(Nc2nc(Nc3ccc(Oc4ccnc(C(=O)NC)c4)cc3)ncc2F)c1] |
| 23. | (7S)-3-fluoro-4-[3-(8-fluoro-1- methyl-2,4-dioxoquinazolin-3-yl)-2- methylphenyl]-7-(2-hydroxypropan- 2-yl)-6,7,8,9-tetrahydro-5H- carbazole-1-carboxamide | 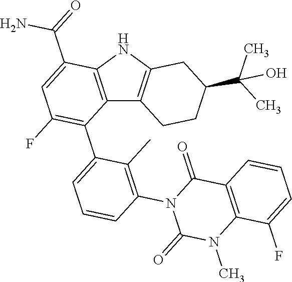 [SMILES: Cc1c(-c2c(F)cc(C(N)=O)c3[nH]c4c(c23)CC[C@H](C(C)(C)O)C4)cccc1-n1c(=O)c2cccc(F)c2n(C)c1=O] |
| 24. | 1-[3-fluoro-4-[7-(5-methyl-1H- imidazol-2-yl)-1-oxo-2,3- dihydroisoindol-4-yl]phenyl]-3-[3- (trifluoromethyl)phenyl]urea | 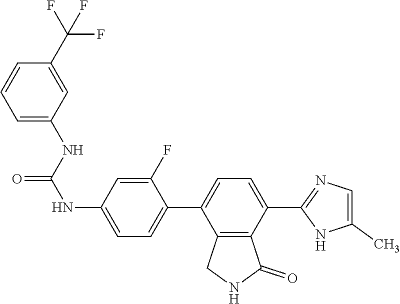 [SMILES: Cc1cnc(-c2ccc(-c3ccc(NC(=O)Nc4cccc(C(F)(F)F)c4)cc3F)c3c2C(=O)NC3)[nH]1] |
| 25. | 9-(1-methylpyrazol-4-yl)-1-(1-prop- 2-enoyl-2,3-dihydroindol-6- yl)benzo[b][1,6]naphthyridin-2-one | 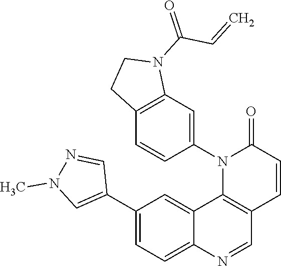 [SMILES: C=CC(=O)N1CCc2ccc(-n3c(=O)ccc4cnc5ccc(-c6cnn(C)c6)cc5c43)cc21] |
| 26. | 7-(2-hydroxypropan-2-yl)-4-[2- methyl-3-(4-oxoquinazolin-3-yl)phe- nyl]-9H-carbazole-1-carboxamide | 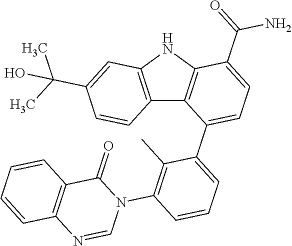 [SMILES: Cc1c(-c2ccc(C(N)=O)c3[nH]c4cc(C(C)(C)O)ccc4c23)cccc1-n1cnc2ccccc2c1=O] |
| 27. | 10-[2-(Hydroxymethyl)-3-[1-methyl- 6-oxo-5-(pyrimidin-4- ylamino)pyridin-3- yl]phenyl]-4,4-dimethyl-7-thia-10- azatricyclo[6.4.0.02,6]dodeca- 1(8),2(6)-dien-9-one | 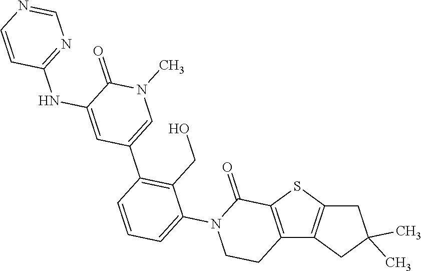 [SMILES: Cn1cc(-c2cccc(N3CCc4c(sc5c4CC(C)(C)C5)C3=O)c2CO)cc(Nc2ccncn2)c1=O] |
| 28. | (S)-5-amino-1-(1-cyanopiperidin-3- yl)-3-(4-(2,4-difluorophenoxy)phe- nyl)-1H-pyrazole-4-carboxamide | 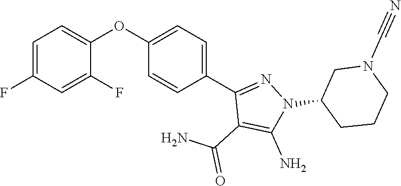 [SMILES: N#CN1CCC[C@H](n2nc(-c3ccc(Oc4ccc(F)cc4F)cc3)c(C(N)=O)c2N)C1] |
| 29. | (S)-4-(3-(1-Acryloylpyrrolidin-2-yl)- 8-aminoimidazo[1,5-a]pyrazin-1-yl)- N-(pyridin-2-yl)benzamide | 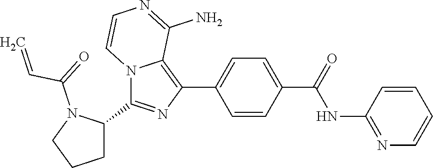 [SMILES: C=CC(=O)N1CCC[C@H]1c1nc(-c2ccc(C(=O)Nc3ccccn3)cc2)c2c(N)nccn12] |
| 30. | (S,E)-4-(8-Amino-3-(1-(4- (dimethylamino)but-2- enoyl)pyrrolidin-2-yl)imidazo[1,5- a]pyrazin-1-yl)-N(pyridin-2- yl)benzamide | 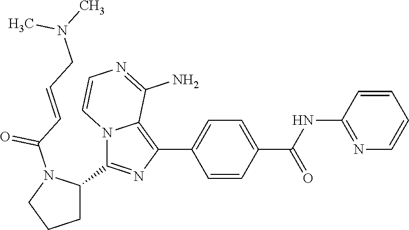 [SMILES: CN(C)C/C=C/C(=O)N1CCC[C@H]1c1nc(-c2ccc(C(=O)Nc3ccccn3)cc2)c2c(N)nccn12] |
| 31. | (S)-4-(8-Amino-3-(1-but-2- ynoylpyrrolidin-2-yl)imidazo[1,5- a]pyrazin-1-yl)-N-(4-methylpyridin- 2-yl)benzamide | 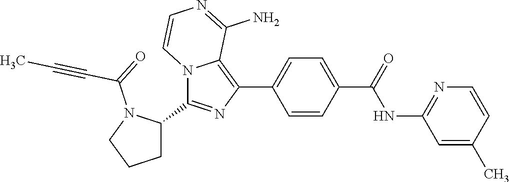 [SMILES: CC#CC(=O)N1CCC[C@H]1c1nc(-c2ccc(C(=O)Nc3cc(C)ccn3)cc2)c2c(N)nccn12] |
| 32. | (S,E)-4-(8-Amino-3-(1-(4-methoxy- but-2-enoyl)pyrrolidin-2-yl)imi- dazo[1,5-a]pyrazin-1-yl)-N- (4-propylpyridin-2-yl)benzamide | 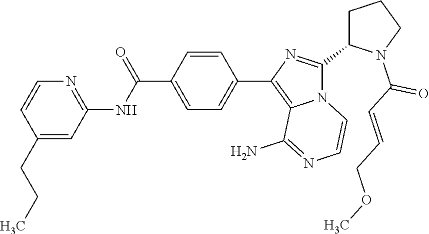 [SMILES: CCCc1ccnc(NC(=O)c2ccc(-c3nc([C@@H]4CCCN4C(=O)/C=C/COC)n4ccnc(N)c34)cc2)c1] |
| 33. | (S)-4-(8-Amino-3-(1-but-2- ynoylpyrrolidin-2-yl)imidazo[1,5-a] pyrazin-1-yl)-N-(4- (trifluoromethyl)pyridin-2- yl)benzamide | 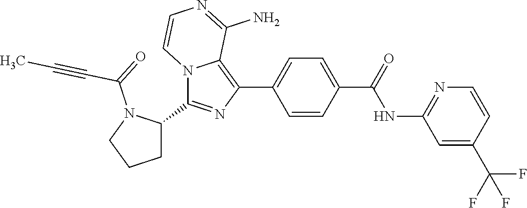 [SMILES: CC#CC(=O)N1CCC[C@H]1c1nc(-c2ccc(C(=O)Nc3cc(C(F)(F)F)ccn3)cc2)c2c(N)nccn12] |
| 34. | (S)-4-(8-Amino-3-(1-but-2- ynoylpyrrolidin-2-yl)imidazo[1,5- a]pyrazin-1-yl)-N-(4,5,6,7- tetrahydrobenzo[d]thiazol-2- yl)benzamde | 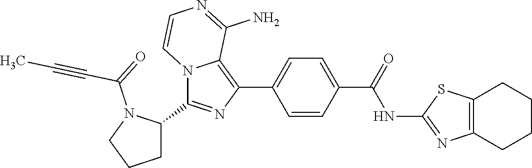 [SMILES: CC#CC(=O)N1CCC[C@H]1c1nc(-c2ccc(C(=O)Nc3nc4c(s3)CCCC4)cc2)c2c(N)nccn12] |
| 35. | (S)-4-(3-(1-acryloylpyrrolidin-2-yl)- 8-aminoimidazo[1,5-a]pyrazin-1- yl)-2-fluoro-N-(pyridin-2- yl)benzamide | 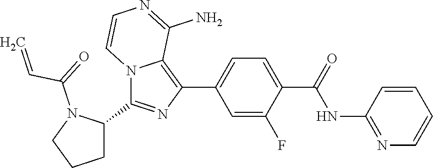 [SMILES: C=CC(=O)N1CCC[C@H]1c1nc(-c2ccc(C(=O)Nc3ccccn3)c(F)c2)c2c(N)nccn12] |
| 36. | (S)-4-(3-(1-Acryloylpyrrolidin-2-yl)- 8-aminoimidazo[1,5-a]pyrazin-1- yl)-2-methoxy-N-(pyridin-2- yl)benzamide | 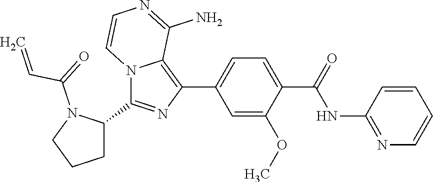 [SMILES: C=CC(=O)N1CCC[C@H]1c1nc(-c2ccc(C(=O)Nc3ccccn3)c(OC)c2)c2c(N)nccn12] |
| 37. | (S,E)-4-(8-Amino-3-(1-(4- (dimethylamino)but-2- enoyl)pyrrolidin-2-yl)imidazo[1,5- a]pyrazin-1-yl)-N-(thiazol-2- yl)benzamide | 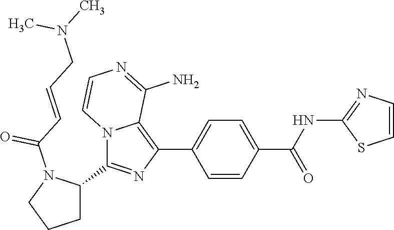 [SMILES: CN(C)C/C=C/C(=O)N1CCC[C@H]1c1nc(-c2ccc(C(=O)Nc3nccs3)cc2)c2c(N)nccn12] |
| 38. | (S)-4-(3-(1-Acryloylpiperidin-2-yl)- 8-aminoimidazo[1,5-a]pyrazin-1-yl)- N-(4-fluoropyridin-2-yl)benzamide | 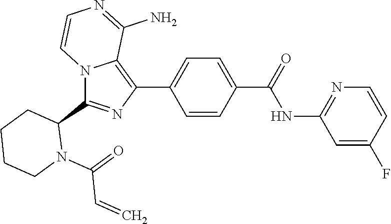 [SMILES: C=CC(=O)N1CCCC[C@H]1c1nc(-c2ccc(C(=O)Nc3cc(F)ccn3)cc2)c2c(N)nccn12] |
| 39. | (S)-4-(3-(1-Acryloylpiperidin-2-yl)- 8-aminoimidazo[1,5-a]pyrazin-1-yl)- N-(4-cyanopyridin-2-yl)benzamide | 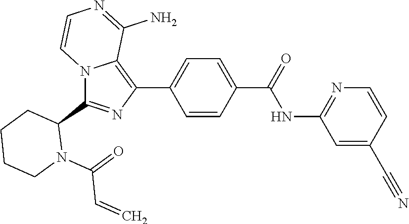 [SMILES: C=CC(=O)N1CCCC[C@H]1c1nc(-c2ccc(C(=O)Nc3cc(C#N)ccn3)cc2)c2c(N)nccn12] |
| 40. | (S)-4-(8-Amino-3-(1- (vinylsulfonyl)piperidin-2- yl)imidazo[1,5-a]pyrazin-1-yl)-N-(4- (trifluoromethyl)pyridin-2- yl)benzamide | 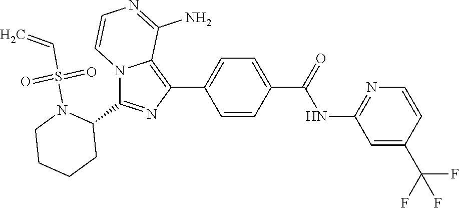 [SMILES: C=CS(=O)(=O)N1CCCC[C@H]1c1nc(-c2ccc(C(=O)Nc3cc(C(F)(F)F)ccn3)cc2)c2c(N)nccn12] |
| 41. | (S)-4-(3-(1-Acryloylpiperidin-2-yl)- 8-aminoimidazo[1,5-a]pyrazin-1-yl)- N-(pyrimidin-2-yl)benzamide | 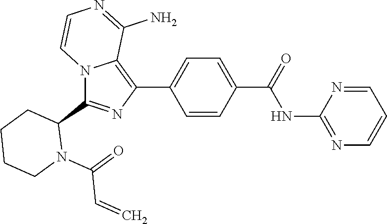 [SMILES: C=CC(=O)N1CCCC[C@H]1c1nc(-c2ccc(C(=O)Nc3ncccn3)cc2)c2c(N)nccn12] |
| 42. | (S)-4-(3-(1-Acryloylpiperidin-2-yl)- 8-aminoimidazo[1,5-a]pyrazin-1- yl)-N-(4-methylpyrimidin-2- yl)benzamide | 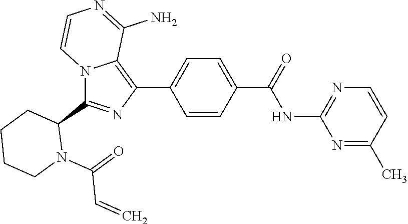 [SMILES: C=CC(=O)N1CCCC[C@H]1c1nc(-c2ccc(C(=O)Nc3nccc(C)n3)cc2)c2c(N)nccn12] |
| 43. | (S)-4-(8-Amino-3-(1-but-2- ynoylpiperidin-2-yl)imidazo[1,5-a] pyrazin-1-yl)-N-(pyrimidin-4- yl)benzamide | 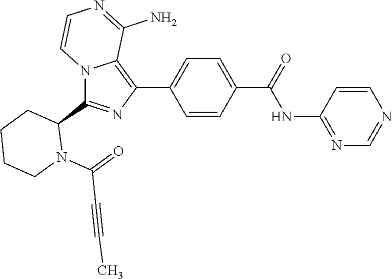 [SMILES: CC#CC(=O)N1CCCC[C@H]1c1nc(-c2ccc(C(=O)Nc3ccncn3)cc2)c2c(N)nccn12] |
| 44. | (S)-4-(8-Amino-3-(1-but-2- ynoylpiperidin-2-yl)imidazo[1,5-a] pyrazin-1-yl)-N-(pyridazin-3- yl)benzamide | 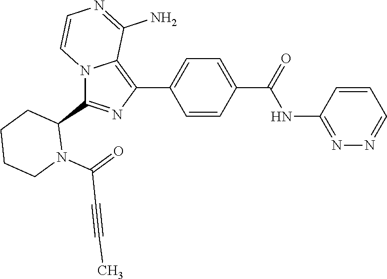 [SMILES: CC#CC(=O)N1CCCC[C@H]1c1nc(-c2ccc(C(=O)Nc3cccnn3)cc2)c2c(N)nccn12] |
| 45. | (S,E)-4-(8-Amino-3-(1-(4-methoxy- but-2-enoyl)piperidin-2-yl)imi- dazo[1,5-a]pyrazin-1-yl)-N-(5- ethylthiazol-2-yl)benzamide | 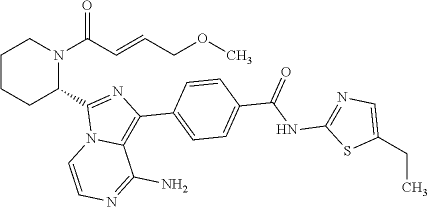 [SMILES: CCc1cnc(NC(=O)c2ccc(-c3nc([C@@H]4CCCCN4C(=O)/C=C/COC)n4ccnc(N)c34)cc2)s1] |
| 46. | (S)-4-(3-(1-Acryloylpiperidin-2-yl)- 8-aminoimidazo[1,5-a]pyrazin-1-yl)- 2-fluoro-N-(4-propylpyridin-2- yl)benzamide | 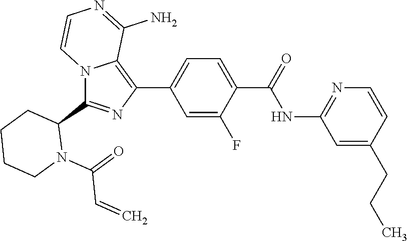 [SMILES: C=CC(=O)N1CCCC[C@H]1c1nc(-c2ccc(C(=O)Nc3cc(CCC)ccn3)c(F)c2)c2c(N)nccn12] |
| 47. | (S,E)-4-(8-Amino-3-(1-(4-(di- methylamino)but-2-enoyl)piperidin- 2-yl)imidazo[1,5-a]pyrazin-1-yl)-2- methoxy-N-(4-propylpyridin-2- yl)benzamide | 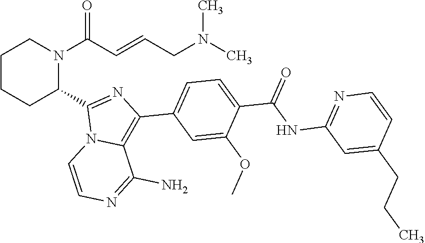 [SMILES: CCCc1ccnc(NC(=O)c2ccc(-c3nc([C@@H]4CCCCN4C(=O)/C=C/CN(C)C)n4ccnc(N)c34)cc2OC)c1] |
| 48. | 4-(8-Amino-3-((S)-1-but-2- ynoylpiperidin-2-yl)imidazo[1,5-a] pyrazin-1-yl)-3-methyl-N-(pyridin-2- yl)benzamide | 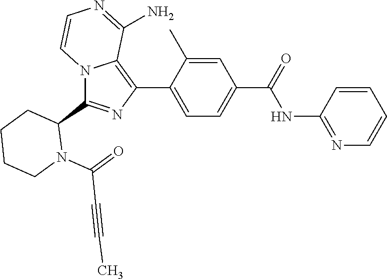 [SMILES: CC#CC(=O)N1CCCC[C@H]1c1nc(-c2ccc(C(=O)Nc3ccccn3)cc2C)c2c(N)nccn12] |
| 49. | 4-(3-(Acrylamidomethyl)-8- aminoimidazo[1,5-a]pyrazin-1-yl)-N- (pyridin-2-yl)benzamide | 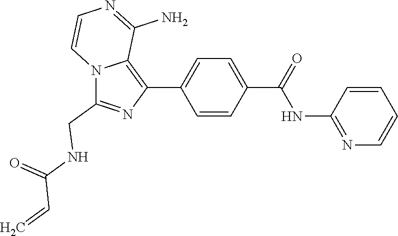 [SMILES: C=CC(=O)NCc1nc(-c2ccc(C(=O)Nc3ccccn3)cc2)c2c(N)nccn12] |
| 50. | (S)-4-(8-Amino-3-(1-but-2-yn- amidoethyl)imidazo[1,5-a]pyrazin-1- yl)-N-(pyridin-2-yl)benzamide | 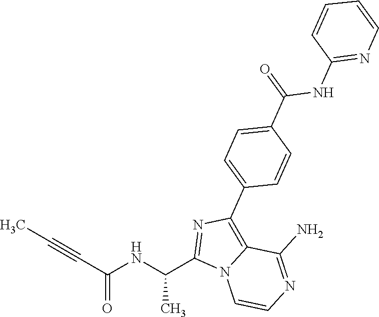 [SMILES: CC#CC(=O)N[C@@H](C)c1nc(-c2ccc(C(=O)Nc3ccccn3)cc2)c2c(N)nccn12] |
| 51. | (S)-S-(2-(2-(8-amino-1-(4-(pyridin- 2-ylcarbamoyl)phenyl)imidazo[1,5- a]pyrazin-3-yl)pyrrolidin-1-yl)-2- oxoethyl)ethanethioate | 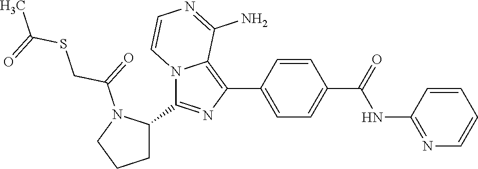 [SMILES: CC(=O)SCC(=O)N1CCC[C@H]1c1nc(-c2ccc(C(=O)Nc3ccccn3)cc2)c2c(N)nccn12] |
| 52. | (S)-4-(8-Amino-3-(1-(4-hydroxy-4- methylpent-2-ynoyl)pyrrolidin-2- yl)imidazo[1,5-a]pyrazin-1-yl)-N(py- ridin-2-yl)benzamide | 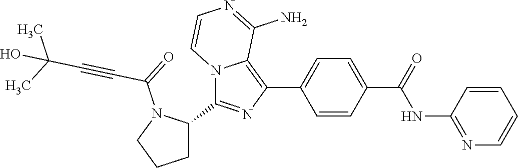 [SMILES: CC(C)(O)C#CC(=O)N1CCC[C@H]1c1nc(-c2ccc(C(=O)Nc3ccccn3)cc2)c2c(N)nccn12] |
| 53. | (S)-4-(8-Amino-3-(1-(6- chloropyrimidine-4-car- bonyl)pyrrolidin-2-yl)imidazo[1,5-a] pyrazin-1-yl)-N-(pyridin-2- yl)benzamide | 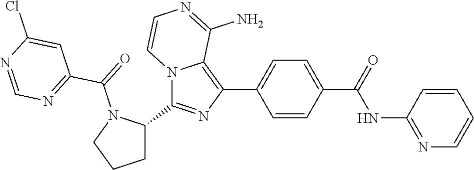 [SMILES: Nc1nccn2c([C@@H]3CCCN3C(=O)c3cc(Cl)ncn3)nc(-c3ccc(C(=O)Nc4ccccn4)cc3)c12] |
| 54. | (S)-4-(8-Amino-3-(1-pent-2- ynoylpyrrolidin-2-yl)imidazo[1,5-a] pyrazin-1-yl)-N-(pyridin-2- yl)benzamide | 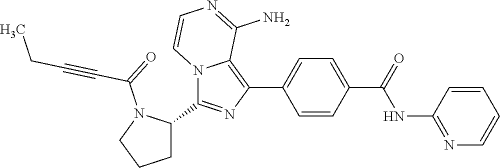 [SMILES: CCC#CC(=O)N1CCC[C@H]1c1nc(-c2ccc(C(=O)Nc3ccccn3)cc2)c2c(N)nccn12] |
| 55. | (S)-4-(8-Amino-3-(1-(3- cyclopropylpropioloyl)pyrrolidin-2- yl)imidazo[1,5-a]pyrazin-1-yl)-N- (pyridin-2-yl)benzamide | 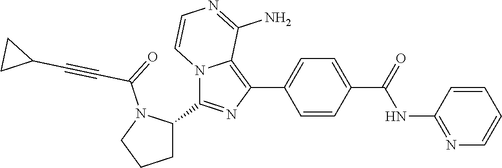 [SMILES: Nc1nccn2c([C@@H]3CCCN3C(=O)C#CC3CC3)nc(-c3ccc(C(=O)Nc4ccccn4)cc3)c12] |
| 56. | (S)-4-(8-Amino-3-(1-hex-2- ynoylpyrrolidin-2-yl)imidazo[1,5-a] pyrazin-1-yl)-N-(pyridin-2- yl)benzamide | 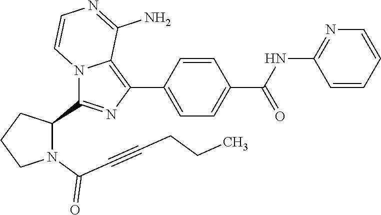 [SMILES: CCCC#CC(=O)N1CCC[C@H]1c1nc(-c2ccc(C(=O)Nc3ccccn3)cc2)c2c(N)nccn12] |
| 57. | 4-(3-(1-Acryloylazepan-2-yl)-8- aminoimidazo[1,5-a]pyrazin-1-yl)- N-(pyridin-2-yl)benzamide | 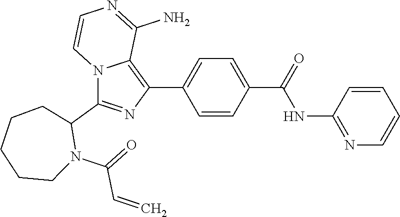 [SMILES: C=CC(=O)N1CCCCCC1c1nc(-c2ccc(C(=O)Nc3ccccn3)cc2)c2c(N)nccn12] |
| 58. | (R)-4-(8-Amino-3-(4-but-2- ynoylmorpholin-3-yl)imidazo[1,5-a] pyrazin-1-yl)-N-(pyridin-2- yl)benzamide | 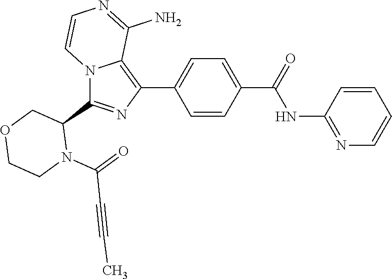 [SMILES: CC#CC(=O)N1CCOC[C@H]1c1nc(-c2ccc(C(=O)Nc3ccccn3)cc2)c2c(N)nccn12] |
| 59. | (S)-4-(8-amino-3-(1-(N-methyl- but-2-ynamido)ethyl)imidazo[1,5- a]pyrazin-1-yl)-N-(4-(trifluoro- methyl)pyridin-2-yl)benzamide | 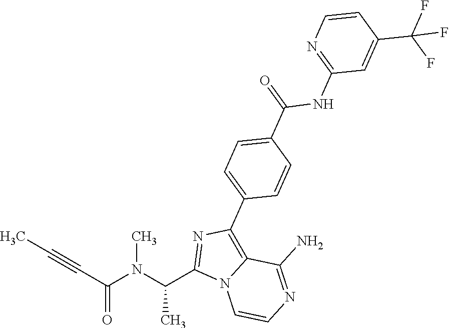 [SMILES: CC#CC(=O)N(C)[C@@H](C)c1nc(-c2ccc(C(=O)Nc3cc(C(F)(F)F)ccn3)cc2)c2c(N)nccn12] |
| 60. | (S)-4-(3-(1-acryloylpyrrolidin-2-yl)- 8-aminoimidazo[1,5-a]pyrazin-1-yl)- N-(4-fluoropyridin-2-yl)benzamide | 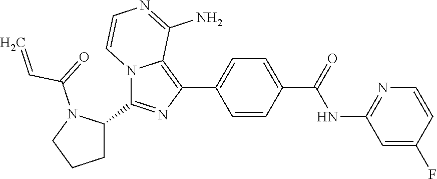 [SMILES: C=CC(=O)N1CCC[C@H]1c1nc(-c2ccc(C(=O)Nc3cc(F)ccn3)cc2)c2c(N)nccn12] |
| 61. | (S)-4-(3-(1-acryloylpyrrolidin-2-yl)- 8-aminoimidazo[1,5-a]pyrazin-1-yl)- N-(4-(pyrrolidin-1-yl)pyridin-2- yl)benzamide | 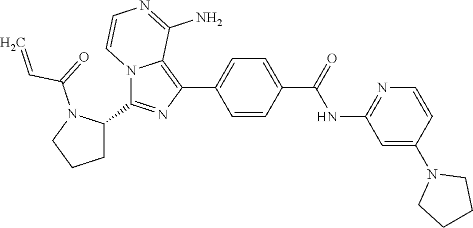 [SMILES: C=CC(=O)N1CCC[C@H]1c1nc(-c2ccc(C(=O)Nc3cc(N4CCCC4)ccn3)cc2)c2c(N)nccn12] |
| 62. | (S)-4-(8-amino-3-(1-but-2- ynoylpiperidin-2-yl)imidazo[1,5- a]pyrazin-1-yl)-N-(4-fluoropyridin- 2-yl)benzamide | 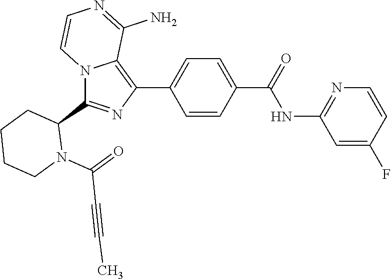 [SMILES: CC#CC(=O)N1CCCC[C@H]1c1nc(-c2ccc(C(=O)Nc3cc(F)ccn3)cc2)c2c(N)nccn12] |
| 63. | (S)-4-(8-amino-3-(1-but-2- ynoylpiperidin-2-yl)imidazo[1,5- a]pyrazin-1-yl)-N-(pyridine-2- yl)benzamide | 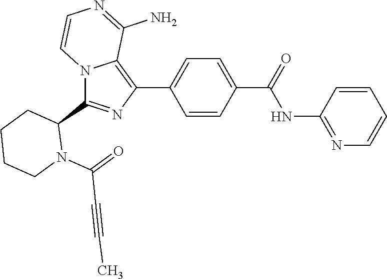 [SMILES: CC#CC(=O)N1CCCC[C@H]1c1nc(-c2ccc(C(=O)Nc3ccccn3)cc2)c2c(N)nccn12] |
| 64. | (S)-4-(3-(1-acryloylpiperidin-2-yl)- 8-aminoimidazo[1,5-a]pyrazin- 1-yl)-N-(pyridine-2-yl)benzamide | 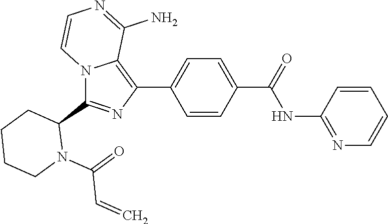 [SMILES: C=CC(=O)N1CCCC[C@H]1c1nc(-c2ccc(C(=O)Nc3ccccn3)cc2)c2c(N)nccn12] |
| 65. | (S)-4-(8-amino-3-(1-but-2- ynoylpyrrolidin-2-yl)imidazo[1,5- a]pyrazin-1-yl)-N-(4-propylpyridin- 2-yl)benzamide | 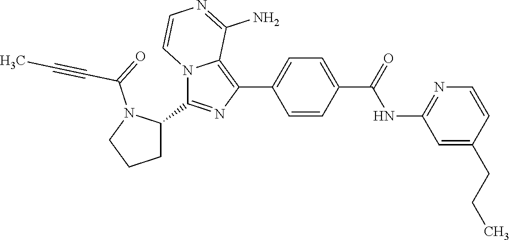 [SMILES: CC#CC(=O)N1CCC[C@H]1c1nc(-c2ccc(C(=O)Nc3cc(CCC)ccn3)cc2)c2c(N)nccn12] |
| 66. | (S,E)-4-(8-amino-3-(1-(4-methoxy- N-methylbut-2-enamido)eth- yl)imidazo[1,5-a]pyrazin- 1-yl)-N-(4-propylpyridin-2- yl)benzamide | 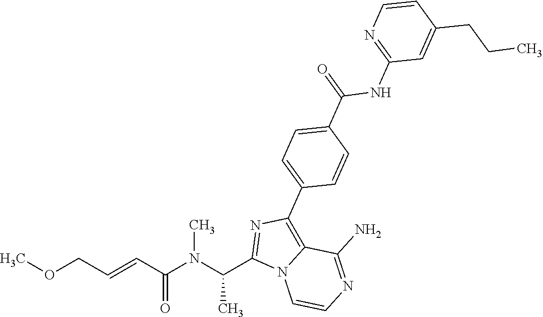 [SMILES: CCCc1ccnc(NC(=O)c2ccc(-c3nc([C@H](C)N(C)C(=O)/C=C/COC)n4ccnc(N)c34)cc2)c1] |
| 67. | (S)-4-(8-amino-3-(1- (vinylsulfonyl)piperidin-2- yl)imidazo[1,5-a]pyrazin-1-yl)-N-(4- propylpyridin-2-yl)benzamide | 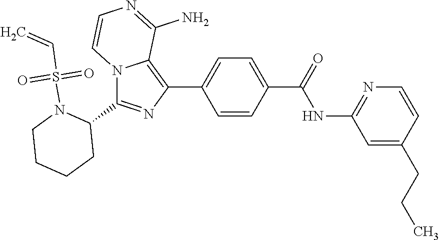 [SMILES: C=CS(=O)(=O)N1CCCC[C@H]1c1nc(-c2ccc(C(=O)Nc3cc(CCC)ccn3)cc2)c2c(N)nccn12] |
| 68. | (S)-4-(3-(1-acryloylpiperidin-2-yl)- 8-aminoimidazo[1,5-a]pyrazin-1-yl)- N-(4-propylpyridin-2-yl)benzamide | 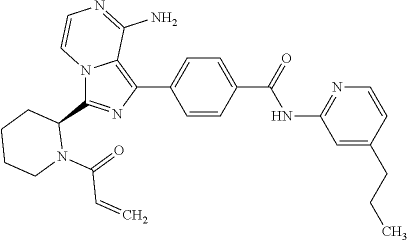 [SMILES: C=CC(=O)N1CCCC[C@H]1c1nc(-c2ccc(C(=O)Nc3cc(CCC)ccn3)cc2)c2c(N)nccn12] |
| 69. | (S)-4-(3-(1-acryloylpyrrolidin-2-yl)- 8-aminoimidazo[1,5-a]pyrazin-1-yl)- N-(4-(trifluoromethyl)pyridin-2- yl)benzamide | 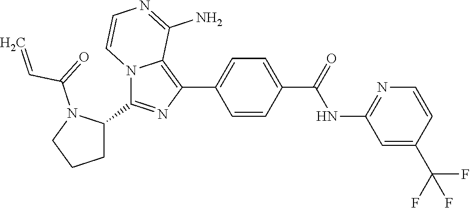 [SMILES: C=CC(=O)N1CCC[C@H]1c1nc(-c2ccc(C(=O)Nc3cc(C(F)(F)F)ccn3)cc2)c2c(N)nccn12] |
| 70. | (S)-4-(8-amino-3-(1-but-2- ynoylpiperidin-2-yl)imidazo[1,5- a]pyrazin-1-yl)-N-(4- (trifluoromethyl)pyridin-2- yl)benzamide | 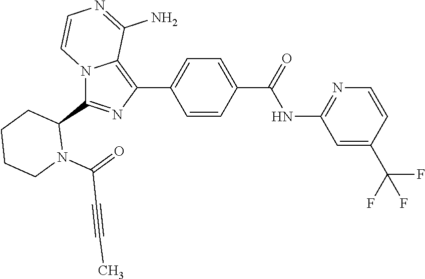 [SMILES: CC#CC(=O)N1CCCC[C@H]1c1nc(-c2ccc(C(=O)Nc3cc(C(F)(F)F)ccn3)cc2)c2c(N)nccn12] |
| 71. | (S)-4-(8-amino-3-(1-but-2- ynoylpiperidin-2-yl)imidazo[1,5- a]pyrazin-1-yl)-N-(4-propylpyridin- 2-yl)benzamide | 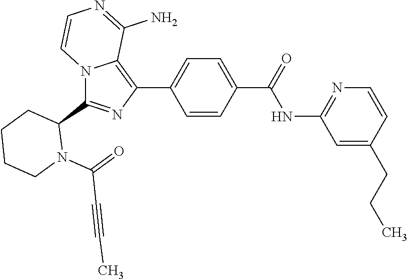 [SMILES: CC#CC(=O)N1CCCC[C@H]1c1nc(-c2ccc(C(=O)Nc3cc(CCC)ccn3)cc2)c2c(N)nccn12] |
| 72. | (S,E)-4-(8-amino-3-(1-(4- (dimethylamino)but-2- enoyl)pyrrolidin-2-yl)imidazo[1,5- a]pyrazin-1-yl)-N-(4-isopropyl- pyridin-2-yl)benzamide | 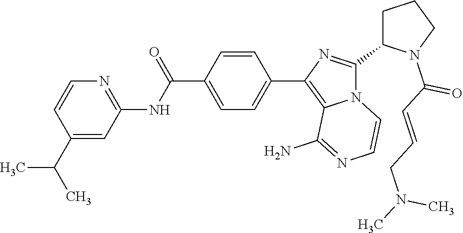 [SMILES: CC(C)c1ccnc(NC(=O)c2ccc(-c3nc([C@@H]4CCCN4C(=O)/C=C/CN(C)C)n4ccnc(N)c34)cc2)c1] |
| 73. | 4-(8-amino-3-((S)-1- (vinylsulfonyl)piperidin-2- yl)imidazo[1,5-a]pyrazin-1-yl)-3- methyl-N-(pyridin-2-yl)benzamide | 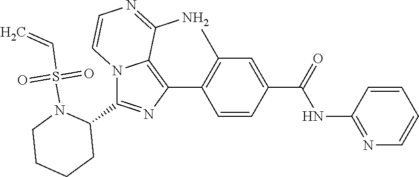 [SMILES: C=CS(=O)(=O)N1CCCC[C@H]1c1nc(-c2ccc(C(=O)Nc3ccccn3)cc2C)c2c(N)nccn12] |
| 74. | (S)-4-(8-amino-3-(1-but-2- ynoylpiperidin-2-yl)imidazo[1,5- a]pyrazin-1-yl)-2-fluoro-N-(4- propylpyridin-2-yl)benzamide | 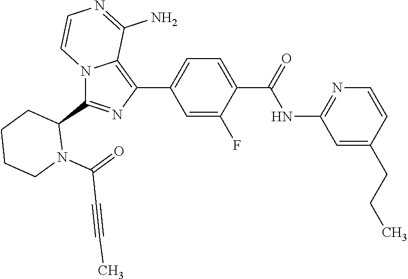 [SMILES: CC#CC(=O)N1CCCC[C@H]1c1nc(-c2ccc(C(=O)Nc3cc(CCC)ccn3)c(F)c2)c2c(N)nccn12] |
| 75. | (S,E)-4-(8-amino-3-(1-(4-methoxy- N-methylbut-2-enamido)eth- yl)imidazo[1,5-a]pyrazin- 1-yl)-N-(4-(trifluoromethyl)pyridin- 2-yl)benzamide | 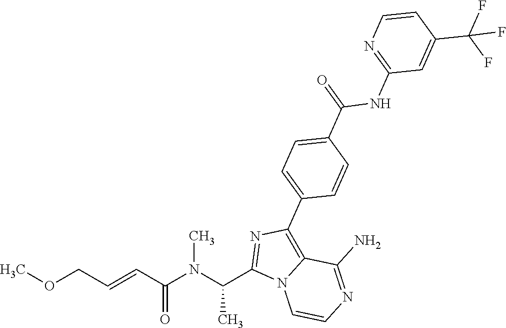 [SMILES: COC/C=C/C(=O)N(C)[C@@H](C)c1nc(-c2ccc(C(=O)Nc3cc(C(F)(F)F)ccn3)cc2)c2c(N)nccn12] |
| 76. | (S,E)-4-(8-amino-3-(1-(4- (dimethylamino)-N-methylbut-2- enamido)ethyl)imidazo[1,5-a]pyra- zin-1-yl)-N-(4-propylpyridin-2- yl)benzamide | 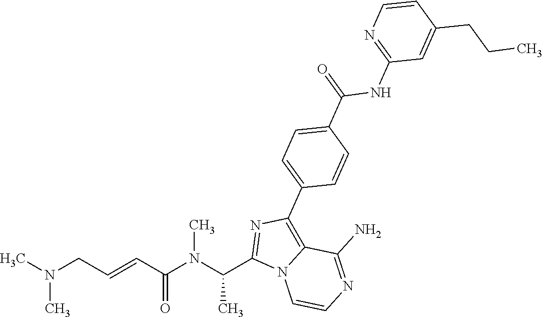 [SMILES: CCCc1ccnc(NC(=O)c2ccc(-c3nc([C@H](C)N(C)C(=O)/C=C/CN(C)C)n4ccnc(N)c34)cc2)c1] |
| 77. | (S,E)-4-(8-amino-3-(1-(4-(pyrroli- din-1-yl)but-2-enoyl)pyrroli- din-2-yl)imidazo[1,5-a]pyrazin- 1-yl)-N-(4-propylpyridin-2- yl)benzamide | 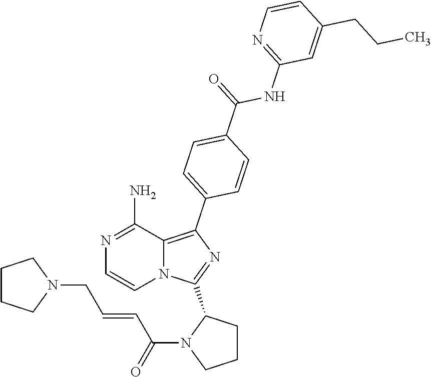 [SMILES: CCCc1ccnc(NC(=O)c2ccc(-c3nc([C@@H]4CCCN4C(=O)/C=C/CN4CCCC4)n4ccnc(N)c34)cc2)c1] |
| 78. | (S,E)-4-(8-amino-3-(1-(4-(di- methylamino)but-2-enoyl)piperidin- 2-yl)imidazo[1,5-a]pyrazin-1-yl)-N- (pyridin-2-yl)benzamide | 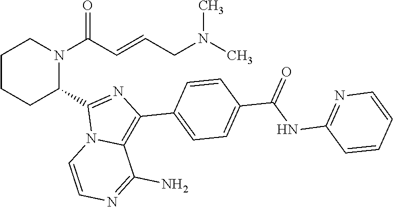 [SMILES: CN(C)C/C=C/C(=O)N1CCCC[C@H]1c1nc(-c2ccc(C(=O)Nc3ccccn3)cc2)c2c(N)nccn12] |
| 79. | (S)-4-(8-amino-3-(1-(2- chloropyrimidine-4-car- bonyl)pyrrolidin-2-yl)imidazo[1,5- a]pyrazin-1-yl)-N-(4-propylpyridin- 2-yl)benzamide | 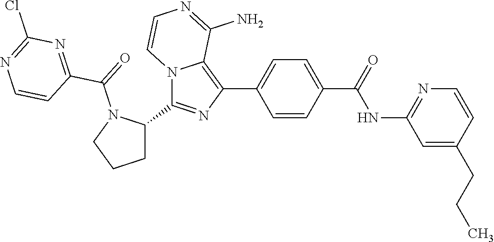 [SMILES: CCCc1ccnc(NC(=O)c2ccc(-c3nc([C@@H]4CCCN4C(=O)c4ccnc(Cl)n4)n4ccnc(N)c34)cc2)c1] |
| 80. | (S)-4-(3-(1-acrylamidoethyl)-8- aminoimidazo[1,5-a]pyrazin-1-yl)- N-(pyridin-2-yl)benzamide | 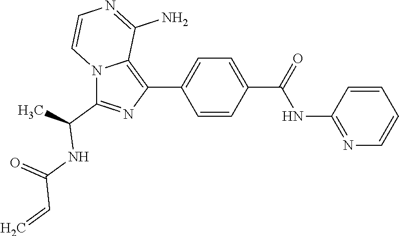 [SMILES: C=CC(=O)N[C@@H](C)c1nc(-c2ccc(C(=O)Nc3ccccn3)cc2)c2c(N)nccn12] |
| 81. | (S)-4-(3-(1-acryloylpyrrolidin-2-yl)- 8-aminoimidazo[1,5-a]pyrazin-1-yl)- N-(thiazol-2-yl)benzamide | 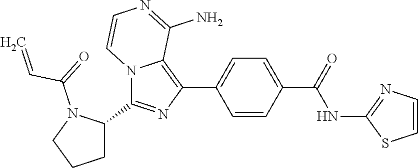 [SMILES: C=CC(=O)N1CCC[C@H]1c1nc(-c2ccc(C(=O)Nc3nccs3)cc2)c2c(N)nccn12] |
| 82. | (S)-4-(8-amino-3-(1-but-2- ynoylpyrrolidin-2-yl)imidazo[1,5- a]pyrazin-1-yl)-N-(4-isopropyl- pyridin-2-yl)benzamide | 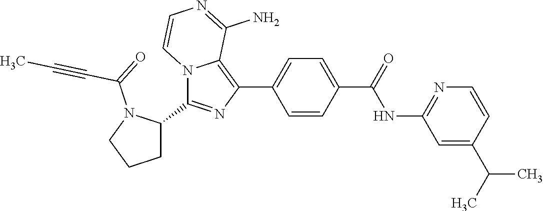 [SMILES: CC#CC(=O)N1CCC[C@H]1c1nc(-c2ccc(C(=O)Nc3cc(C(C)C)ccn3)cc2)c2c(N)nccn12] |
| 83. | (S)-4-(8-amino-3-(1-(2-chloro- pyrimidine-4-carbonyl)piperidin- 2-yl)imidazo[1,5-a]pyrazin-1-yl)-N- (4-propylpyridin-2-yl)benzamide | 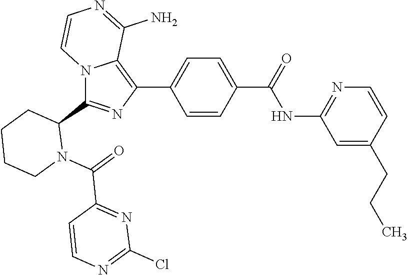 [SMILES: CCCc1ccnc(NC(=O)c2ccc(-c3nc([C@@H]4CCCCN4C(=O)c4ccnc(Cl)n4)n4ccnc(N)c34)cc2)c1] |
| 84. | (S,E)-4-(8-amino-3-(1-(4-methoxy- but-2-enoyl)piperidin-2-yl)imi- dazo[1,5-a]pyrazin-1-yl)-N-(4- (trifluoromethyl)pyridin-2-yl) benzamide | 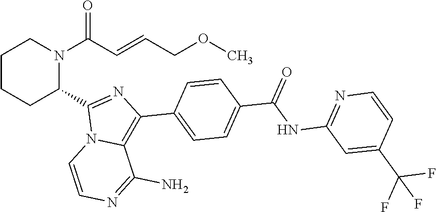 [SMILES: COC/C=C/C(=O)N1CCCC[C@H]1c1nc(-c2ccc(C(=O)Nc3cc(C(F)(F)F)ccn3)cc2)c2c(N)nccn12] |
| 85. | (S)-4-(3-(1-acryloylpiperidin-2-yl)- 8-aminoimidazo[1,5-a]pyrazin-1-yl)- N-(4-(trifluoromethyl)pyridin-2- yl)benzamide | 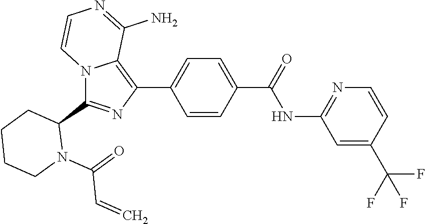 [SMILES: C=CC(=O)N1CCCC[C@H]1c1nc(-c2ccc(C(=O)Nc3cc(C(F)(F)F)ccn3)cc2)c2c(N)nccn12] |
| 86. | (S)-4-(8-amino-3-(1-but-2- ynoylpiperidin-2-yl)imidazo[1,5- a]pyrazin-1-yl)-2-methoxy-N-(4- propylpyridin-2-yl)benzamide | 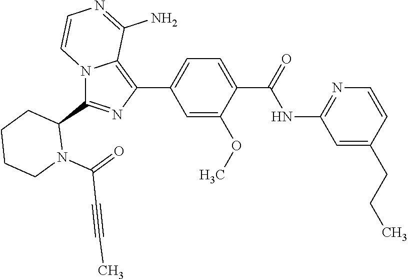 [SMILES: CC#CC(=O)N1CCCC[C@H]1c1nc(-c2ccc(C(=O)Nc3cc(CCC)ccn3)c(OC)c2)c2c(N)nccn12] |
| 87. | (S,E)-4-(8-amino-3-(1-(4-methoxy- but-2-enoyl)piperidin-2-yl)imi- dazo[1,5-a]pyrazin-1-yl)-2- methoxy-N-(4-propylpyridin-2- yl)benzamide | 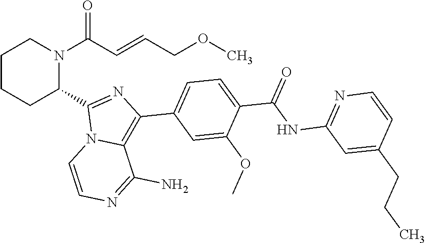 [SMILES: CCCc1ccnc(NC(=O)c2ccc(-c3nc([C@@H]4CCCCN4C(=O)/C=C/COC)n4ccnc(N)c34)cc2OC)c1] |
| 88. | (S)-4-(8-amino-3-(1-(2-chloro- pyrimidine-4-carbonyl)piperidin- 2-yl)imidazo[1,5-a]pyrazin-1-yl)-N- (4-(trifluoromethyl)pyridin-2- yl)benzamide | 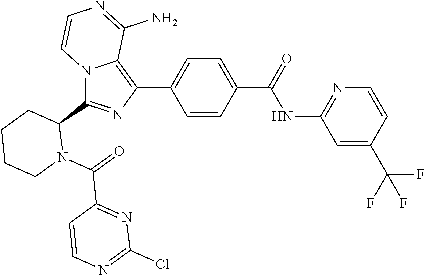 [SMILES: Nc1nccn2c([C@@H]3CCCCN3C(=O)c3ccnc(Cl)n3)nc(-c3ccc(C(=O)Nc4cc(C(F)(F)F)ccn4)cc3)c12] |
| 89. | (S)-4-(8-amino-3-(1-but-2- ynoylpiperidin-2-yl)imidazo[1,5- a]pyrazin-1-yl)-N-(5-ethylthiazol-2- yl)benzamide | 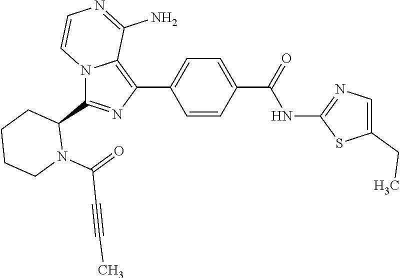 [SMILES: CC#CC(=O)N1CCCC[C@H]1c1nc(-c2ccc(C(=O)Nc3ncc(CC)s3)cc2)c2c(N)nccn12] |
| 90 | (S)-4-(3-(1-acryloylpiperidin-2-yl)- 8-aminoimidazo[1,5-a]pyrazin-1-yl)- N-(5-ethylthiazol-2-yl)benzamide | 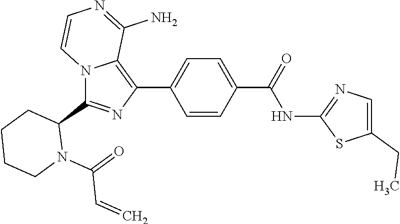 [SMILES: C=CC(=O)N1CCCC[C@H]1c1nc(-c2ccc(C(=O)Nc3ncc(CC)s3)cc2)c2c(N)nccn12] |
| 91. | (S)-4-(8-amino-3-(1-(2-chloro- pyrimidine-4-carbonyl)piperidin- 2-yl)imidazo[1,5-a]pyrazin-1-yl)-N- (5-ethylthiazol-2-yl)benzamide | 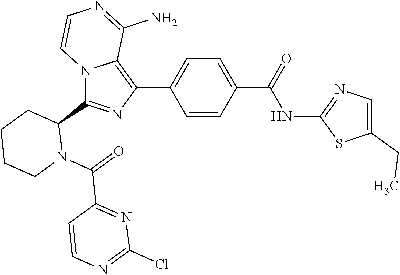 [SMILES: CCc1cnc(NC(=O)c2ccc(-c3nc([C@@H]4CCCCN4C(=O)c4ccnc(Cl)n4)n4ccnc(N)c34)cc2)s1] |
| 92. | (R,E)-4-(8-amino-3-(4-(4-methoxy- but-2-enoyl)morpholin-3-yl)imi- dazo[1,5-a]pyrazin-1-yl)-N- (pyridin-2-yl)benzamide | 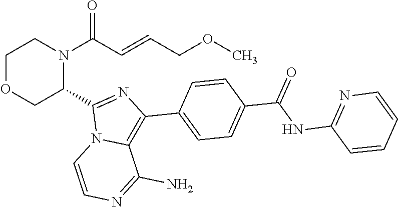 [SMILES: COC/C=C/C(=O)N1CCOC[C@H]1c1nc(-c2ccc(C(=O)Nc3ccccn3)cc2)c2c(N)nccn12] |
| 93. | (S,E)-4-(8-amino-3-(1-(4-methoxy- but-2-enoyl)piperidin-2-yl)imi- dazo[1,5-a]pyrazin-1-yl)-N- (4-propylpyridin-2-yl)benzamide | 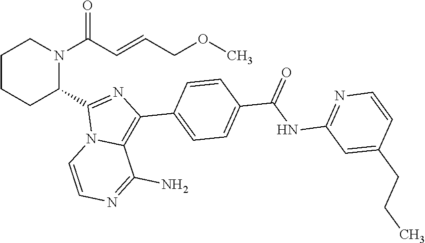 [SMILES: CCCc1ccnc(NC(=O)c2ccc(-c3nc([C@@H]4CCCCN4C(=O)/C=C/COC)n4ccnc(N)c34)cc2)c1] |
| 94. | (S)-4-(3-(1-acryloylpyrrolidin-2-yl)- 8-aminoimidazo[1,5-a]pyrazin-1-yl)- N-(4-cyanopyridin-2-yl)benzamide | 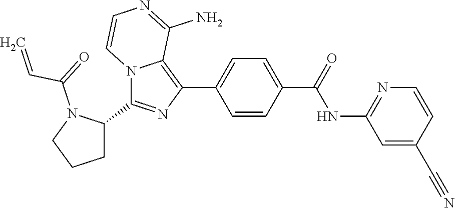 [SMILES: C=CC(=O)N1CCC[C@H]1c1nc(-c2ccc(C(=O)Nc3cc(C#N)ccn3)cc2)c2c(N)nccn12] |
| 95. | (S)-4-(8-amino-3-(1-but-2- ynoylpyrrolidin-2-yl)imidazo[1,5- a]pryazin-1-yl)-N-(4-methoxy- pyridin-2-yl)benzamide | 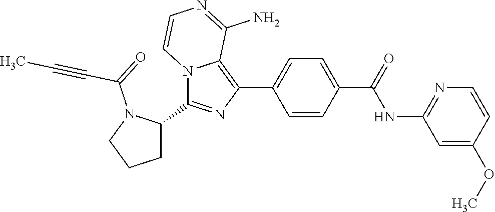 [SMILES: CC#CC(=O)N1CCC[C@H]1c1nc(-c2ccc(C(=O)Nc3cc(OC)ccn3)cc2)c2c(N)nccn12] |
| 96. | (S)-4-(3-(1-acryloylpyrrolidin-2-yl)- 8-aminoimidazo[1,5-a]pyrazin-1-yl)- N-(4-methylpyridin-2-yl)benzamide | 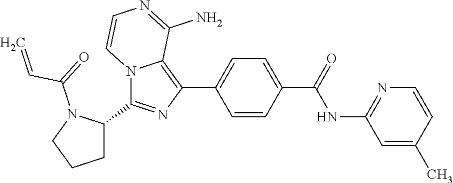 [SMILES: C=CC(=O)N1CCC[C@H]1c1nc(-c2ccc(C(=O)Nc3cc(C)ccn3)cc2)c2c(N)nccn12] |
| 97. | (S)-4-(3-(1-acryloylpyrrolidin-2-yl)- 8-aminoimidazo[1,5-a]pyrazin-1-yl)- N-(4-propylpyridin-2-yl)benzamide | 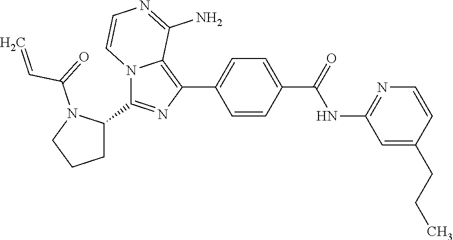 [SMILES: C=CC(=O)N1CCC[C@H]1c1nc(-c2ccc(C(=O)Nc3cc(CCC)ccn3)cc2)c2c(N)nccn12] |
| 98. | (S)-4-(3-(1-acryloylpyrrolidin-2-yl)- 8-aminoimidazo[1,5-a]pyrazin-1-yl)- N-(4-ethylpyridin-2-yl)benzamide | 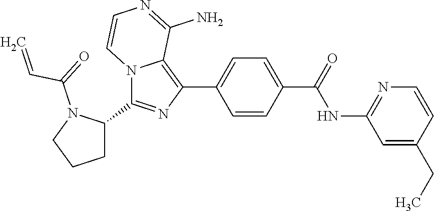 [SMILES: C=CC(=O)N1CCC[C@H]1c1nc(-c2ccc(C(=O)Nc3cc(CC)ccn3)cc2)c2c(N)nccn12] |
| 99. | (S,E)-4-(8-amino-3-(1-(4- (dimethylamino)but-2- enoyl)pyrrolidin-2-yl)imidazo[1,5- a]pyrazin-1-yl)-N-(pyridin-2- yl)benzamide | 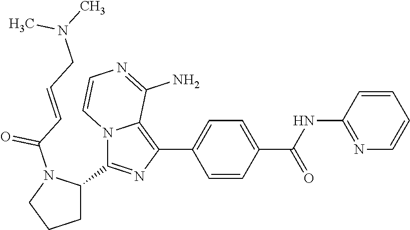 [SMILES: CN(C)C/C=C/C(=O)N1CCC[C@H]1c1nc(-c2ccc(C(=O)Nc3ccccn3)cc2)c2c(N)nccn12] |
| 100. | (S,E)-4-(8-amino-3-(1-(4-methoxy- but-2-enoyl)pyrrolidin-2-yl)imi- dazo[1,5-a]pyrazin-1-yl)-N-(4- (trifluoromethyl)pyridin-2- yl)benzamide | 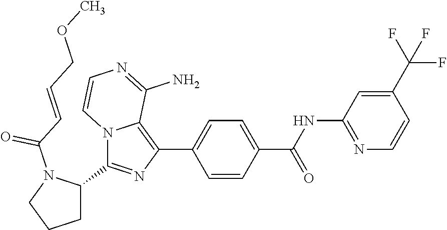 [SMILES: COC/C=C/C(=O)N1CCC[C@H]1c1nc(-c2ccc(C(=O)Nc3cc(C(F)(F)F)ccn3)cc2)c2c(N)nccn12] |
| 101. | (S)-4-(8-amino-3-(1-(2- chloropyrimidine-4-car- bonyl)pyrrolidin-2-yl)imidazo[1,5- a]pyrazin-1-yl)-N-(4-methylpyridin- 2-yl)benzamide | 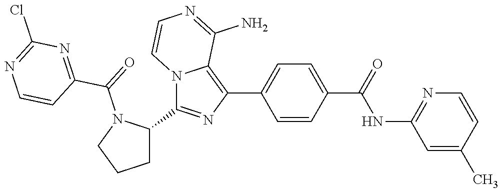 [SMILES: Cc1ccnc(NC(=O)c2ccc(-c3nc([C@@H]4CCCN4C(=O)c4ccnc(Cl)n4)n4ccnc(N)c34)cc2)c1] |
| 102. | (S)-4-(8-amino-3-(1-but-2- ynoylpyrrolidin-2-yl)imidazo[1,5-a] pyrazin-1-yl)-N-(4-cyanopyridin-2- yl)benzamide | 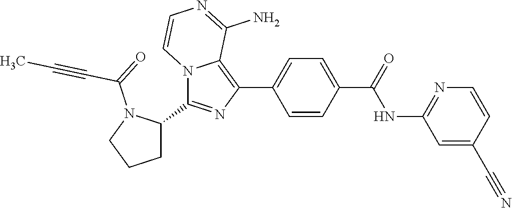 [SMILES: CC#CC(=O)N1CCC[C@H]1c1nc(-c2ccc(C(=O)Nc3cc(C#N)ccn3)cc2)c2c(N)nccn12] |
| 103. | (S)-4-(8-amino-3-(1-but-2- ynoylpyrrolidin-2-yl)imidazo[1,5- a]pyrazin-1-yl)-N-(4-ethylpyridin-2- yl)benzamide | 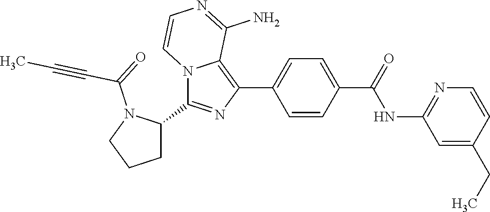 [SMILES: CC#CC(=O)N1CCC[C@H]1c1nc(-c2ccc(C(=O)Nc3cc(CC)ccn3)cc2)c2c(N)nccn12] |
| 104. | (S)-4-(8-amino-3-(1-but-2- ynoylpyrrolidin-2-yl)imidazo[1,5- a]pyrazin-1-yl)-N-(4-phenylpyridin- 2-yl)benzamide | 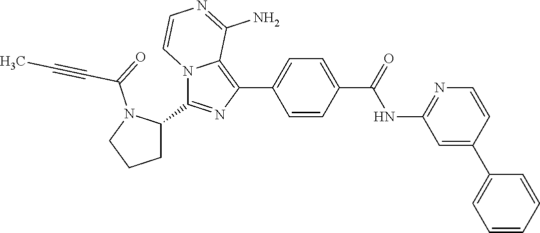 [SMILES: CC#CC(=O)N1CCC[C@H]1c1nc(-c2ccc(C(=O)Nc3cc(-c4ccccc4)ccn3)cc2)c2c(N)nccn12] |
| 105. | (S)-4-(3-(1-acryloylpyrrolidin-2-yl)- 8-aminoimidazo[1,5-a]pyrazin-1- yl)N-(4-phenylpyridin-2- yl)benzamide | 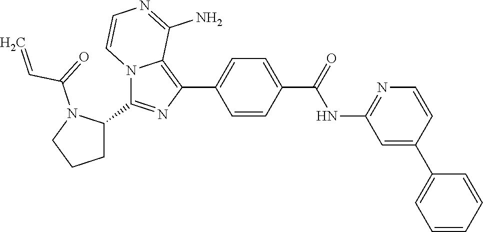 [SMILES: C=CC(=O)N1CCC[C@H]1c1nc(-c2ccc(C(=O)Nc3cc(-c4ccccc4)ccn3)cc2)c2c(N)nccn12] |
| 106. | (R,E)-1-(3-(4-amino-3-(4- phenoxyphenyl)-1H-pyrazolo[3,4- d]pyrimidin-1-yl)piperidin-1-yl)-4- (dimethylamino)but-2-en-1-one | 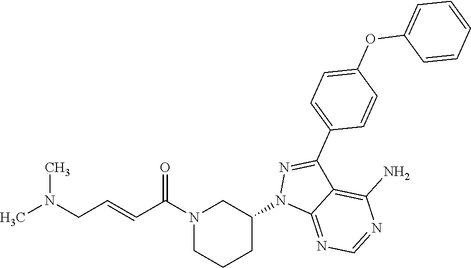 [SMILES: CN(C)C/C=C/C(=O)N1CCC[C@@H](n2nc(-c3ccc(Oc4ccccc4)cc3)c3c(N)ncnc32)C1] |
| 107. | (E)-1-(3-(4-amino-3-(4- phenoxyphenyl)-1H-pyrazolo[3,4- d]pyrimidin-1-yl)piperidin-1-yl)-4- morpholinobut-2-en-1-one | 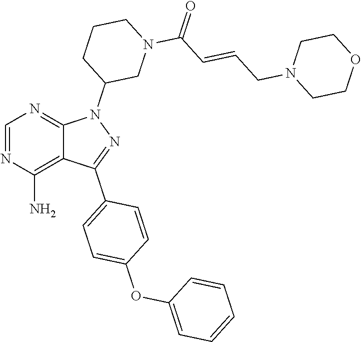 [SMILES: Nc1ncnc2c1c(-c1ccc(Oc3ccccc3)cc1)nn2C1CCCN(C(=O)/C=C/CN2CCOCC2)C1] |
| 108. | 1-(4-(4-amino-3-(4-phenoxyphenyl)- 1H-pyrazolo[3,4-d]pyrimidin-1- yl)piperidin-1-yl)prop-2-en-1-one | 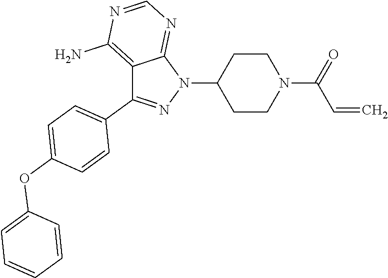 [SMILES: C=CC(=O)N1CCC(n2nc(-c3ccc(Oc4ccccc4)cc3)c3c(N)ncnc32)CC1] |
| 109. | (E)-1-(4-(4-amino-3-(4- phenoxyphenyl)-1H-pyrazolo[3,4- d]pyrimidin-1-yl)piperidin-1-yl)-4- (dimethylamino)but-2-en-1-one | 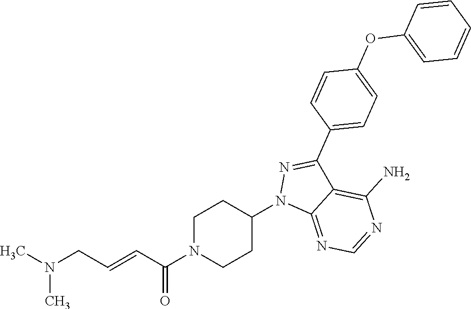 [SMILES: CN(C)C/C=C/C(=O)N1CCC(n2nc(-c3ccc(Oc4ccccc4)cc3)c3c(N)ncnc32)CC1] |
| 110. | (E)-N-((1s,4s)-4-(4-amino-3-(4- phenoxyphenyl)-1H-pyrazolo[3,4- d]pyrimidin-1-yl)cyclohexyl)-4- (dimethylamino)but-2-enamide | 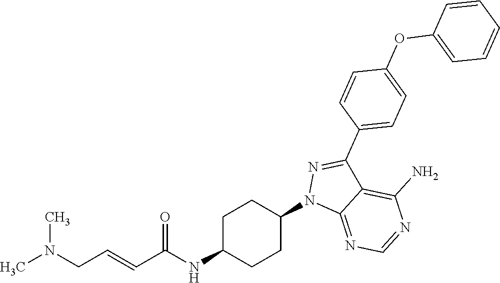 [SMILES: CN(C)C/C=C/C(=O)N[C@H]1CC[C@@H](n2nc(-c3ccc(Oc4ccccc4)cc3)c3c(N)ncnc32)CC1] |
| 111. | 1-(4-(4-amino-3-(4-phenoxyphenyl)- 1H-pyrazolo[3,4-d]pyrimidin-1- yl)piperidin-1-yl)prop-2-en-1-one | 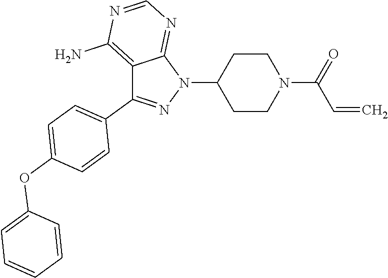 [SMILES: C=CC(=O)N1CCC(n2nc(-c3ccc(Oc4ccccc4)cc3)c3c(N)ncnc32)CC1] |
| 112. | N-((1r,4r)-4-(4-amino-3-(4- phenoxyphenyl)-1H-pyrazolo[3,4- d]pyrimidin-1-yl)cyclohexyl)acryl- amide | 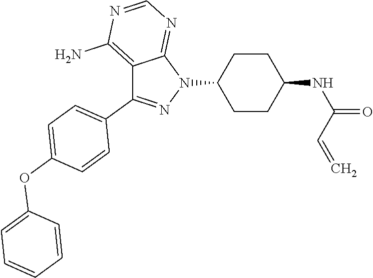 [SMILES: C=CC(=O)N[C@H]1CC[C@H](n2nc(-c3ccc(Oc4ccccc4)cc3)c3c(N)ncnc32)CC1] |
| 113. | (E)-1-((R)-2-((4-amino-3-(4- phenoxyphenyl)-1H-pyrazolo[3,4- d]pyrimidin-1-yl)methyl)pyrrolidin- 1-yl)-4-(dimethylamino)but- 2-en-1-one | 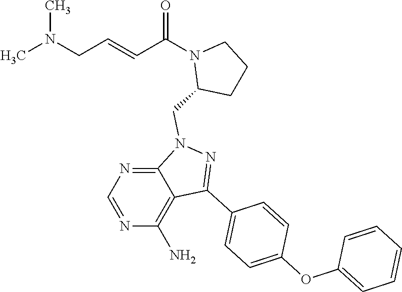 [SMILES: CN(C)C/C=C/C(=O)N1CCC[C@@H]1Cn1nc(-c2ccc(Oc3ccccc3)cc2)c2c(N)ncnc21] |
| 114. | (E)-1-((S)-2-((4-amino-3-(4- phenoxyphenyl)-1H-pyrazolo[3,4- d]pyrimidin-1-yl)methyl)pyrrolidin- 1-yl)-4-(dimethylamino)but- 2-en-1-one | 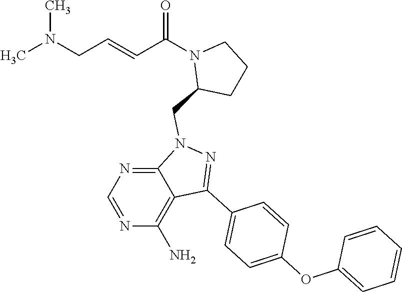 [SMILES: CN(C)C/C=C/C(=O)N1CCC[C@H]1Cn1nc(-c2ccc(Oc3ccccc3)cc2)c2c(N)ncnc21] |
| 115. | 1-((R)-2-((4-amino-3-(4- phenoxyphenyl)-1H-pyrazolo[3,4- d]pyrimidin-1-yl)methyl)pyrrolidin- 1-yl)prop-2-en-1-one | 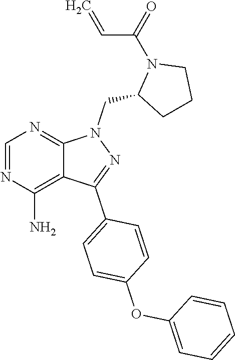 [SMILES: C=CC(=O)N1CCC[C@@H]1Cn1nc(-c2ccc(Oc3ccccc3)cc2)c2c(N)ncnc21] |
| 116. | 1-((S)-2-((4-amino-3-(4- phenoxyphenyl)-1H-pyrazolo[3,4- d]pyrimidin-1-yl)methyl)pyrrolidin- 1-yl)prop-2-en-1-one | 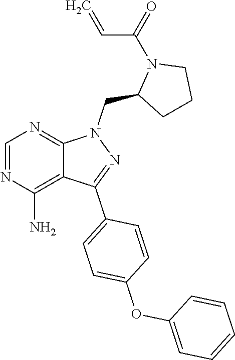 [SMILES: C=CC(=O)N1CCC[C@H]1Cn1nc(-c2ccc(Oc3ccccc3)cc2)c2c(N)ncnc21] |
| 117. | 1-((R)-2-((4-amino-3-(4- phenoxyphenyl)-1H-pyrazolo[3,4- d]pyrimidin-1-yl)methyl)pyrrolidin- 1-yl)but-2-yn-1-one | 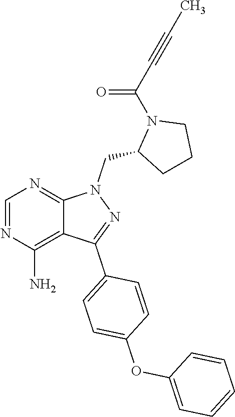 [SMILES: CC#CC(=O)N1CCC[C@@H]1Cn1nc(-c2ccc(Oc3ccccc3)cc2)c2c(N)ncnc21] |
| 118. | 1-((S)-2-((4-amino-3-(4- phenoxyphenyl)-1H-pyrazolo[3,4- d]pyrimidin-1-yl)methyl)pyrrolidin- 1-yl)but-2-yn-1-one | 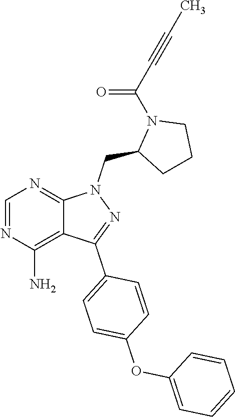 [SMILES: CC#CC(=O)N1CCC[C@H]1Cn1nc(-c2ccc(Oc3ccccc3)cc2)c2c(N)ncnc21] |
| 119. | 1-((R)-3-(4-amino-3-(4- phenoxyphenyl)-1H-pyrazolo[3,4- d]pyrimidin-1-yl)piperidin-1-yl)but- 2-yn-1-one | 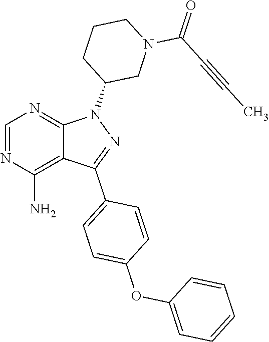 [SMILES: CC#CC(=O)N1CCC[C@@H](n2nc(-c3ccc(Oc4ccccc4)cc3)c3c(N)ncnc32)C1] |
| 120. | (E)-N-((1r,4r)-4-(4-amino-3-(4- phenoxyphenyl)-1H-pyrazolo[3,4- d]pyrimidin-1-yl)cyclohexyl-4- (dimethylamino))but-2-enamide | 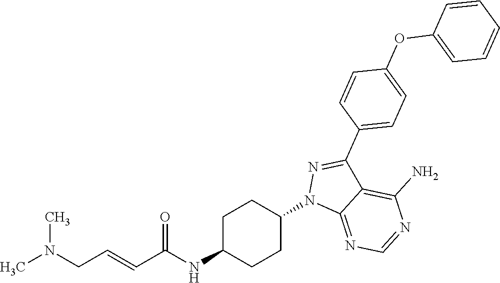 [SMILES: CN(C)C/C=C/C(=O)N[C@H]1CC[C@H](n2nc(-c3ccc(Oc4ccccc4)cc3)c3c(N)ncnc32)CC1] |
| 121. | N-(2-(4-amino-3-(4-phenoxyphenyl)- 1H-pyrazolo[3,4-d]pyrimidin-1- yl)ethyl)-N-methylacrylamide | 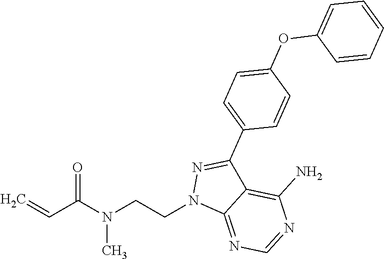 [SMILES: C=CC(=O)N(C)CCn1nc(-c2ccc(Oc3ccccc3)cc2)c2c(N)ncnc21] |
| 122. | (E)-1-(4-(4-amino-3-(4- phenoxyphenyl)-1H-pyrazolo[3,4- d]pyrimidin-1-yl)-4-morpholino)but- 2-en-1-one | 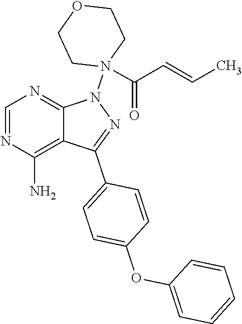 |
| 123. | (E)-1-(2-((S)-2-((4-amino-3-(4- phenoxyphenyl)-1H-pyrazolo[3,4- d]pyrimidin-1-yl)methyl)pyrrolidin- 1-yl)morpholino)but-2-en-1-one | 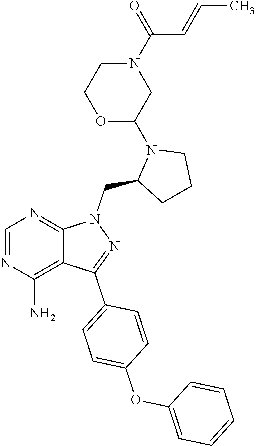 [SMILES: C/C=C/C(=O)N1CCOC(N2CCC[C@H]2Cn2nc(-c3ccc(Oc4ccccc4)cc3)c3c(N)ncnc32)C1] |
| 124. | N-((1s,4s)-4-(4-amino-3-(4- phenoxyphenyl)-1H-pyrazolo[3,4- d]pyrimidin-1-yl)cyclohexyl)but-2- ynamide | 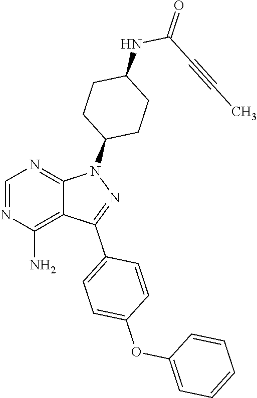 [SMILES: CC#CC(=O)N[C@H]1CC[C@@H](n2nc(-c3ccc(Oc4ccccc4)cc3)c3c(N)ncnc32)CC1] |
| 125. | N-(2-(4-amino-3-(4-phenoxyphenyl)- 1H-pyrazolo[3,4-d]pyrimidin-1- yl)ethyl)acrylamide | 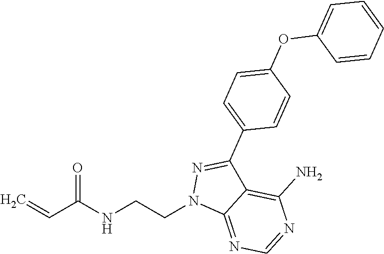 [SMILES: C=CC(=O)NCCn1nc(-c2ccc(Oc3ccccc3)cc2)c2c(N)ncnc21] |
| 126. | (E)-1-((R)-3-(4-amino-3-(4- phenoxyphenyl)-1H-pyrazolo[3,4- d]pyrimidin-1-yl)piperidin-1-yl)-4- morpholinobut-2-en-1-one | 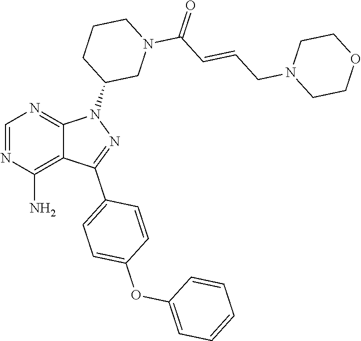 [SMILES: Nc1ncnc2c1c(-c1ccc(Oc3ccccc3)cc1)nn2[C@@H]1CCCN(C(=O)/C=C/CN2CCOCC2)C1] |
| 127. | (E)-N-((1s,4s)-4-(4-amino-3-(4- phenoxyphenyl)-1H-pyrazolo[3,4- d]pyrimidin-1-yl)cyclohexyl)-4- morpholinobut-2-enamide | 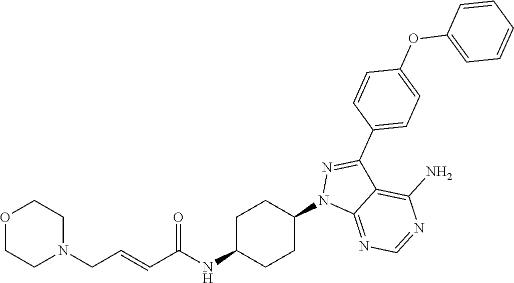 [SMILES: Nc1ncnc2c1c(-c1ccc(Oc3ccccc3)cc1)nn2[C@H]1CC[C@@H](NC(=O)/C=C/CN2CCOCC2)CC1] |
| 128. | 1-(4-(((6-amino-5-(4- phenoxyphenyl)pyrimidin-4- yl)amino)methyl)-4-fluoropiperidin- 1-yl)prop-2-en-1-one | 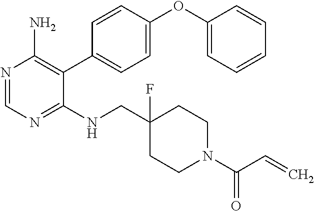 [SMILES: C=CC(=O)N1CCC(F)(CNc2ncnc(N)c2-c2ccc(Oc3ccccc3)cc2)CC1] |
| 129. | N-[3-[[5-fluoro-2-[4-(2- methoxyethoxy)anilino]pyrimidin-4- yl]amino]phenyl]prop-2-enamide | 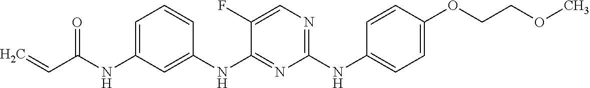 [SMILES: C=CC(=O)Nc1cccc(Nc2nc(Nc3ccc(OCCOC)cc3)ncc2F)c1] |
| 130. | 6-amino-9-[(3R)-1-but-2- ynoylpyrrolidin-3-yl]-7-(4- phenoxyphenyl)purin-8-one | 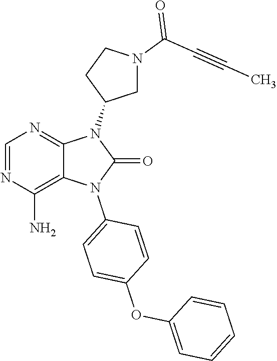 [SMILES: CC#CC(=O)N1CC[C@@H](n2c(=O)n(-c3ccc(Oc4ccccc4)cc3)c3c(N)ncnc32)C1] |
| 131. | (7S)-2-(4-phenoxyphenyl)-7-(1-prop- 2-enoylpiperidin-4-yl)-4,5,6,7- tetrahydropyrazolo[1,5-a]pyrimidine- 3-carboxamide | 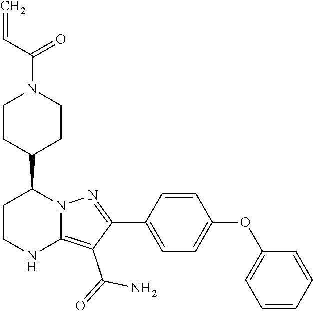 [SMILES: C=CC(=O)N1CCC([C@@H]2CCNc3c(C(N)=O)c(-c4ccc(Oc5ccccc5)cc4)nn32)CC1] |
| 132. | Orelabrutinib (2-(4-phenoxyphenyl)- 6-(1-prop-2-enoylpiperidin-4- yl)pyridine-3-carboxamide | 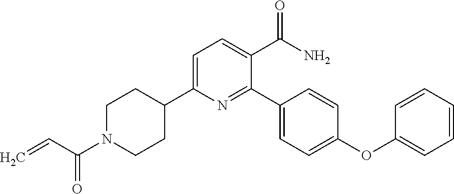 [SMILES: C=CC(=O)N1CCC(c2ccc(C(N)=O)c(-c3ccc(Oc4ccccc4)cc3)n2)CC1] |
| 133. | Remibrutinib (N-[3-[6-amino-5-[2- [methyl(prop-2-enoyl) amino]ethoxy]pyrimidin-4-yl]-5- fluoro-2-methylphenyl]-4-cyclo- propyl-2-fluorobenzamide) | 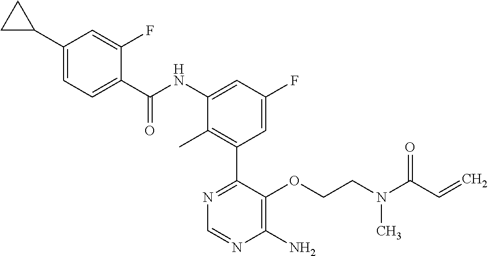 [SMILES: C=CC(=O)N(C)CCOc1c(N)ncnc1-c1cc(F)cc(NC(=O)c2ccc(C3CC3)cc2F)c1C] |
| 134. | Loxo-305 (1H-Pyrazole-4-carbox- amide, 5-amino-3-[4-[[[(5-fluoro-2- methoxybenzoyl)amino]methyl]phe- nyl]-1-[(1S)-2,2,2-trifluoro-1- methylethyl]) | 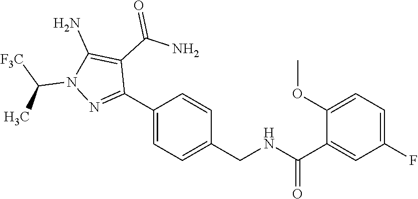 [SMILES: COc1ccc(F)cc1C(=O)NCc1ccc(-c2nn([C@@H](C)C(F)(F)F)c(N)c2C(N)=O)cc1] |
| 135. | TG-1701 (4-amino-1-[(3R)-1-but-2- ynoylpyrrolidin-3-yl]-3-[4-(2,6- difluorophenoxy)phenyl]-6H- pyrrolo[2,3-d]pyridazin-7-one) | 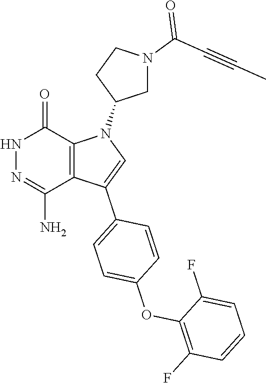 [SMILES: CC#CC(=O)N1CC[C@@H](n2cc(-c3ccc(Oc4c(F)cccc4F)cc3)c3c(N)n[nH]c(=O)c32)C1] |
| 136. | N-((1s,4s)-4-(4-amino-3-(4- phenoxyphenyl)-1H-pyrazolo[3,4- d]pyrimidin-1-yl)cyclo- hexyl)acrylamide | 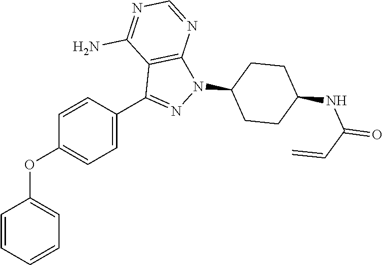 [SMILES: C=CC(=O)N[C@H]1CC[C@@H](n2nc(-c3ccc(Oc4ccccc4)cc3)c3c(N)ncnc32)CC1] |
| 137. | N-((1r,4r)-4-(4-amino-3-(4- phenoxyphenyl)-1H-pyrazolo[3,4- d]pyrimidin-1-yl)cyclohexyl)but-2- ynamide | 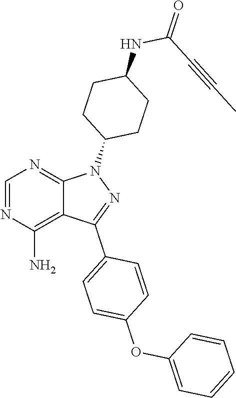 [SMILES: CC#CC(=O)N[C@H]1CC[C@H](n2nc(-c3ccc(Oc4ccccc4)cc3)c3c(N)ncnc32)CC1] |
| 138. | (E)-N-((1r,4r)-4-(4-amino-3-(4- phenoxyphenyl)-1H-pyrazolo[3,4- d]pyrimidin-1-yl)cyclohexyl)-4- morpholinobut-2-enamide | 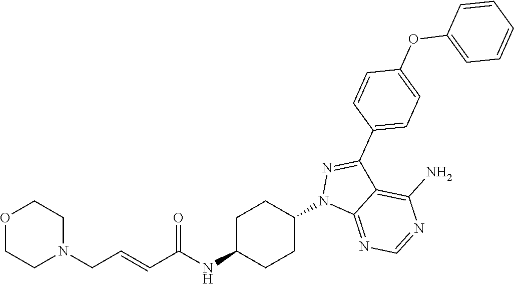 [SMILES: Nc1ncnc2c1c(-c1ccc(Oc3ccccc3)cc1)nn2[C@H]1CC[C@H](NC(=O)/C=C/CN2CCOCC2)CC1] |

In an embodiment, the BTK inhibitor or a pharmaceutically acceptable salt thereof is a reversible BTK inhibitor. In an embodiment, the BTK inhibitor or a pharmaceutically acceptable salt thereof is an irreversible covalent BTK inhibitor.

The present disclosure also relates to a use of a BTK inhibitor compound for treating an ophthalmic condition in a human in need thereof, wherein the ophthalmic condition includes: ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The BTK inhibitor compound is provided in an amount effective to treat the ophthalmic condition in the human. In an embodiment, the BTK inhibitor compound is a compound listed in Table 1 or a pharmaceutically acceptable salt thereof.

The present disclosure further relates to a use of a BTK inhibitor compound for reducing inflammation in an eye of a human in need thereof. The BTK inhibitor compound is provided in an amount effective to reduce inflammation in the eye of the human. In an embodiment, the BTK inhibitor compound is a compound listed in Table 1 or a pharmaceutically acceptable salt thereof.

The present disclosure additionally relates to a use of a BTK inhibitor compound for reducing an immune response in a human in need thereof having an ophthalmic condition. The immune response includes an innate immune response, an adaptive immune response, or both. The ophthalmic condition includes ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The BTK inhibitor compound is provided in an amount effective to reduce the immune response in the human having an ophthalmic condition. In an embodiment, the BTK inhibitor compound is a compound listed in Table 1 or a pharmaceutically acceptable salt thereof.

The present disclosure also relates to a use of a pharmaceutical composition as described herein comprising the BTK inhibitor compound for treating an ophthalmic condition in a human in need thereof, wherein the ophthalmic condition includes: ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The pharmaceutical composition is provided in an amount effective to treat the ophthalmic condition in the human. In an embodiment, the BTK inhibitor compound is a compound listed in Table 1 or a pharmaceutically acceptable salt thereof.

The present disclosure further relates to a use of a pharmaceutical composition as described herein comprising the BTK inhibitor compound for reducing inflammation in an eye of a human in need thereof. The pharmaceutical composition is provided in an amount effective to reduce inflammation in the eye of the human. In an embodiment, the BTK inhibitor compound is a compound listed in Table 1 or a pharmaceutically acceptable salt thereof.

The present disclosure additionally relates to a use of a pharmaceutical composition as described herein comprising the BTK inhibitor compound for reducing an immune response in a human in need thereof having an ophthalmic condition. The immune response includes an innate immune response, an adaptive immune response, or both. The ophthalmic condition includes ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The pharmaceutical composition is provided in an amount effective to reduce the immune response in the human having an ophthalmic condition. In an embodiment, the BTK inhibitor compound is a compound listed in Table 1 or a pharmaceutically acceptable salt thereof.

### Nanoparticle Compositions

The present disclosure includes a pharmaceutical composition comprising nanoparticles comprising the BTK inhibitor compound. In an embodiment, the BTK inhibitor compound is provided in the form of nanoparticles comprising the BTK inhibitor compound. In an embodiment, the BTK inhibitor compound is administered as nanoparticles comprising the BTK inhibitor compound. In an embodiment, the BTK inhibitor compound is administered as a pharmaceutical composition as described herein. In an embodiment, the BTK inhibitor compound is administered as a pharmaceutical composition comprising nanoparticles comprising the BTK inhibitor compound. The present disclosure includes a pharmaceutical composition comprising nanoparticles comprising: a BTK inhibitor, one or more surfactants, and a pharmaceutically acceptable excipient. In an embodiment, the BTK inhibitor compound is a compound listed in Table 1 or a pharmaceutically acceptable salt thereof. In an embodiment, the BTK inhibitor compound is 1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof.

In an embodiment, the nanoparticles further comprise a polymer. The polymer is selected from the group consisting of chitosan, gelatin, sodium alginate, albumin, poly-L-lactide (PLLA), poly(lactic acid) (PLA), poly(glycolic acid)(PGA), poly(lactic co-glycolic acid) (PLGA), polycaprolactone, poly(lactide co-caprolactone), poly(methyl methacrylates), poloxamer, poly(ethylene glycol) (PEG), PEG-PLLA, PEG-PLGA, poly(methyl vinyl ether/maleic anhydride), cellulose acetate phthalate, and combinations thereof.

In an embodiment, the BTK inhibitor is encapsulated in the nanoparticles.

In an embodiment, the polymer is poly(lactic co-glycolic acid) (PLGA).

In an embodiment, PLGA has an average molecular weight of about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, 140 kDa, or 150 kDa.

In an embodiment, PLGA has lactic acid/glycolic acid ratio of 5:95, 10:90; 15:85; 20:80, 25:75, 30:70; 35:65, 40:60, 45:55, 50:50, 55:45, 60:40, 65:35, 70:30, 75:25, 80:20, 85:15, 80:19, or 95:5.

In an embodiment, the surfactant is selected from the group consisting of polysorbate, polyvinyl alcohol, methyl cellulose, gelatin, albumin, poloxamer, ethyl cellulose, crosslinked polyacrylic acid polymer, tocopheryl polyethylene glycol succinate (TPGS), sodium cholate, lipids, stearic acid, and combinations thereof.

In an embodiment, the surfactant is tocopheryl polyethylene glycol succinate (TPGS).

In an embodiment, the nanoparticles further comprise a stabilizer selected from the group consisting of PVP (Povidone), PVA (Polyvinyl alcohol), PEG (Polyethylene glycol), HPMC (Hypromellose), HPC (Hydroxypropyl cellulose), HEC (Hydroxyethyl cellulose), NaCMC (Carboxymethylcellulose sodium), SD (Docusate sodium), SLS (Sodium lauryl sulfate), PEI (Polyethylene imine), TPGS (D-α-tocopheryl polyethylene glycol succinate), PEO (Polyethylene oxide) and PPO (Polypropylene oxide).

In an embodiment, the nanoparticles further comprise a hydrogel.

In an embodiment, the hydrogel is selected from the group consisting of poly(propylene oxide), poly(ethylene oxide), poloxamers (pluronics), chitosan, gelatin, cellulose derivatives, glycol chitin, poly(N-isopropylacrylamide) (PNIPAAm), PEG-PLGA-PEG, poly(D, L-lactide)-poly(ethyleneglycol)-poly(D,L-lactide) (PDLLA-PEG-PDLLA), and combinations thereof.

### Particle Size and Morphology

In some embodiments, the nanoparticles may have a spherical shape. In some embodiments, the nanoparticles may have cylindrical shape.

In some embodiments, the nanoparticles may have a wide variety of non-spherical shapes. The non-spherical shaped nanoparticles can be used to alter uptake by phagocytic cells and thereby clearance by the reticuloendothelial system. In some embodiments, the non-spherical nanoparticles may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks. Additional shapes beyond those are also within the scope of the definition for “non-spherical” shapes.

In some embodiments, the particle has a median particle size less than 1000 nm. In some embodiments, the median particle size ranges from about 1 nm to about 1000 nm. In some embodiments, the median particle size ranges from about 1 nm to about 500 nm. In some embodiments, the median particle size ranges from about 1 nm to about 250 nm. In some embodiments, the median particle size ranges from about 1 nm to about 150 nm. In some embodiments, the median particle size ranges from about 1 nm to about 100 nm. In some embodiments, the median particle size ranges from about 1 nm to about 50 nm. In some embodiments, the median particle size ranges from about 1 nm to about 25 nm. In some embodiments, the median particle size ranges from about 1 nm to about 10 nm. In some embodiments, the particle has a median particle size selected from the group consisting of about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, and about 1000 nm. In some embodiments, the nanoparticle has a median particle size of 500 nm. In some embodiments, the nanoparticle has a median particle size of 250 nm.

In an embodiment, the nanoparticles have a median particle size about 5 nm, about 10 nm, about 15 nm, about 20 nm, about 25 nm, about 30 nm, about 35 nm, about 40 nm, about 45 nm, about 50 nm, about 55 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm.

In an embodiment, the nanoparticles have a median particle size less than about 50 nm, less than about 60 nm, less than about 70 nm, less than about 80 nm, less than about 90 nm, less than about 100 nm, less than about 110 nm, less than about 120 nm, less than about 130 nm, less than about 140 nm, less than about 150 nm, less than about 160 nm, less than about 170 nm, less than about 180 nm, less than about 190 nm, less than about 200 nm, less than about 210 nm, less than about 220 nm, or less than about 230 nm.

In an embodiment, the nanoparticles have a median particle size in a range from about 5 nm to about 200 nm, from about 10 nm to about 190 nm, from about 15 nm to about 180 nm, from about 20 nm to about 175 nm, from about 25 nm to about 170 nm, from about 30 nm to about 165 nm, from about 35 nm to about 160 nm, from about 40 nm to about 155 nm, from about 45 nm to about 150 nm, from about 50 nm to about 145 nm, from about 55 nm to about 140 nm, from about 60 nm to about 135 nm, from about 65 nm to about 130 nm, from about 70 nm to about 125 nm, from about 75 nm to about 120 nm, from about 80 nm to about 115 nm, from about 85 nm to about 110 nm, or from about 90 nm to about 100 nm.

In an embodiment, the nanoparticles have a PDI about 0.05, about 0.10, about 0.15, about 0.20, about 0.25, about 0.30, about 0.35, about 0.40, about 0.45, about 0.50, about 0.55, about 0.60, about 0.65, about 0.70, about 0.75, about 0.80, about 0.85, about 0.90, about 0.95, or about 1.0.

In an embodiment, the nanoparticles have a PDI less than about 0.05, less than about 0.10, less than about 0.15, less than about 0.20, less than about 0.25, less than about 0.30, less than about 0.35, less than about 0.40, less than about 0.45, less than about 0.50, less than about 0.55, less than about 0.60, less than about 0.65, less than about 0.70, less than about 0.75, less than about 0.80, less than about 0.85, less than about 0.90, less than about 0.95, or less than about 1.00.

In an embodiment, the nanoparticles have a PDI in a range from about 0.05 to about 1.00, from about 0.06 to about 0.9, from about 0.07 to about 0.8, from about 0.08 to about 0.7, from about 0.09 to about 0.6, or from about 0.1 to about 0.5.

In some embodiments, the nanoparticles have a PDI from about 0.05 to about 0.15, from about 0.06 to about 0.14, from about 0.07 to about 0.13, from about 0.08 to about 0.12, or from about 0.09 to about 0.11. In some embodiments, the nanoparticles have a PDI of about 0.05, about 0.06, about 0.07, about 0.08, about 0.09, about 0.10, about 0.11, about 0.12, about 0.13, about 0.14, or about 0.15

### Polymer

In some embodiments, the nanoparticles further comprise a polymer selected from the group consisting of chitosan, gelatin, sodium alginate, albumin, poly-L-lactide (PLLA), poly(lactic acid) (PLA), poly(glycolic acid)(PGA), poly(lactic co-glycolic acid) (PLGA), polycaprolactone, poly(lactide co-caprolactone), poly(methyl methacrylates), poloxamer, poly(ethylene glycol) (PEG), PEG-PLLA, PEG-PLGA, poly(methyl vinyl ether/maleic anhydride), cellulose acetate phthalate, and combinations thereof.

In some embodiments, the polymer is a lipid selected from the group consisting of lipid, polymer-lipid conjugate, carbohydrate-lipid conjugate, peptide-lipid conjugate, protein-lipid conjugate, and combinations thereof. In some embodiments, the lipid may include one or more of the following: phospholipids such as phosphatidylcholines, phosphatidylserines, phosphatidylinositides, phosphatidylethanolamines, phosphatidylglycerols, phosphatidic acids; sphingolipids such as sphingomyelins, ceramides, phytoceramides, cerebrosides; sterols such as cholesterol, desmosterol, lathosterol, stigmasterol, zymosterol, diosgenin, and combinations thereof.

In some embodiments, the polymer is conjugated with a lipid to form a polymer-lipid conjugate, wherein the polymers conjugated to polar head groups of the lipid may include polyethylene glycol, polyoxazolines, polyglutamines, polyasparagines, polyaspartamides, polyacrylamides, polyacrylates, polyvinylpyrrolidone, or polyvinylmethylether.

In some embodiments, the polymer is a carbohydrate-lipid conjugate, wherein the carbohydrate is conjugated to the lipid and may include monosaccharides (glucose, fructose, glyceraldehydes etc.), disaccharides, oligosaccharides or polysaccharides such as glycosaminoglycan (hyaluronic acid, keratan sulfates, heparin sulfate or chondroitin sulfate), carrageenan, microbial exopolysaccharides, alginate, chitosan, pectins, chitin, cellulose, or starch.

In one embodiment, the phospholipid is selected from the group consisting of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC), 1,2-dimyristoyl-sn-glycero-3-phosphocholine (DMPC), 1,2-dimyristoyl-sn-glycero-3-phosphorylglycerol (DMPG), 1,2-distearoyl-sn-glycero-3-phosphoethanolamine (DSPE), 1,2-dioleoyl-sn-glycero-3-phosphocholine (DOPC), 1,2-dioleoyl-sn-glycero-3-phosphoethanolamine (DOPE), 1,2-dipalmitoyl-sn-glycero-3-phospho-(1′-rac-glycerol) (DPPG), 1,2-distearoyl-sn-glycero-3-phosphocholine (DSPC), distearoylphosphoethanolamine conjugated with polyethylene glycol (DSPE-PEG), phosphatidylserine (PS), phosphatidylethanolamine (PE), phosphatidylglycerol (PG), phosphatidylcholine (PC), and combinations thereof. In an embodiment, the particle comprises a lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, PG, and combinations thereof.

In some embodiments, the lipid is selected from the group consisting of 1,2-dipalmitoyl-sn-glycero-3-phospho-(1′-rac-glycerol) (DPPG); 1,2-distearoyl-sn-glycero-3-phosphoglycerol, sodium salt (DSPG); 1,2-dimyristoyl-sn-glycero-3-phospho-L-serine sodium salt (DMPS, 14:0 PS); 1,2-dipalmitoyl-sn-glycero-3-phosphoserine, sodium salt (DPPS, 16:0 PS); 1,2-distearoyl-sn-glycero-3-phospho-L-serine (sodium salt) (DSPS, 18:0 PS); 1,2-dimyristoyl-sn-glycero-3-phosphate, sodium salt (DMPA, 14:0 PA); 1,2-dipalmitoyl-sn-glycero-3-phosphate, sodium salt (DPPA, 16:0 PA); 1,2-distearoyl-sn-glycero-3-phosphate, sodium salt (DSPA, 18:0); 1′,3′-bis[1,2-dipalmitoyl-sn-glycero-3-phospho]-glycerol sodium salt (16:0 cardiolipin); 1,2-dimyristoyl-sn-glycero-3-phosphoethanolamine (DMPE, 12:0 PE); 1,2-dipalmitoyl-sn-glycero-3-phosphoethanolamine (DPPE, 16:0); 1,2-diarachidyl-sn-glycero-3-phosphoethanolamine (20:0 PE); 1-stearoyl-2-linoleoyl-sn-glycero-3-phosphoethanolamine; 1,2-diheptadecanoyl-sn-glycero-3-phosphocholine (17:0 PC); 1,2-dinonadecanoyl-sn-glycero-3-phosphocholine (19:0 PC); 1,2-diarachidoyl-sn-glycero-3-phosphocholine (20:0 PC); 1,2-diheneicosanoyl-sn-glycero-3-phosphocholine (21:0 PC); 1,2-dibehenoyl-sn-glycero-3-phosphocholine (22:0 PC); 1,2-ditricosanoyl-sn-glycero-3-phosphocholine (23:0 PC); 1,2-dilignoceroyl-sn-glycero-3-phosphocholine (24:0 PC); 1-myristoyl-2-stearoyl-sn-glycero-3-phosphocholine (14:0-18:0 PC); 1-stearoyl-2-palmitoyl-sn-glycero-3-phosphocholine (16:0-18:0 PC); and combinations thereof.

In some embodiments, the polymer is a biocompatible polymer. In some embodiments, the polymer is a biodegradable polymer.

In some embodiments, the polymer is selected from the group consisting of PDMS (poly (dimethyl siloxane) (PDMS)), polydioxanone, poliglecaprone, polypropylene, polyvinylidene fluoride, polyethylene terephthalate, polyethylene including ultra-high-molecular-weight polyethylene (UHMWPE), cross-linked UHMWPE, low density polyethylene (LDPE), high density polyethylene (HDPE), polyketones, polystyrene, polyvinyl chloride, poly (meth) acrylamides, polyetheretherketone (PEEK), poly(methyl methacrylate), polyester including poly(lactic acid-co-glycolic acid) (PLGA), polyglycolic acid (PGA), polylactic acid (PLA), polycaprolactone (PCL), poly(trimethylene carbonate), poly (alpha-esters), polyurethanes, poly(allylamine hydrochloride), poly(ester amides), poly (ortho esters), polyanyhydrides, poly (anhydride-co-imide), cross-linked polyanhydrides, pseudo poly(amino acids), poly (alkylcyanoacrylates), polyphosphoesters, polyphosphazenes, chitosan, collagen, gelatin, natural or synthetic poly(amino acids), elastin, elastin-linked polypeptides, albumin, fibrin, polysiloxanes, polycarbosiloxanes, polysilazanes, polyalkoxysiloxanes, polysaccharides, cross-linkable polymers, thermoresponsive polymers, thermo-thinning polymers, thermo-thickening polymers, block co-polymers comprising polyethylene glycol, and combinations thereof.

In some embodiments, the polymer is selected from the group consisting of PGA, PLA, PLGA, polydioxanone, polycaprolactone, and combinations thereof.

In some embodiments, the polymer is present at a weight percentage by the total weight of the nanoparticles selected from the group consisting of about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, or about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, and about 99.0 wt. %. In some embodiments, the polymer is present at a weight percentage by the total weight of the nanoparticles in a range from about 1 wt. % to about 99 wt. %, from about 10.0 wt. % to about 95.0 wt. %, from about 50.0 wt. % to about 95.0 wt. %, from about 25.0 wt. % to about 90.0 wt. % or from about 75.0 wt. % to about 90.0 wt. %.

### Hydrogel

In some embodiments, the nanoparticles further comprise a hydrogel selected from the group consisting of poly(propylene oxide), poly(ethylene oxide), poloxamers (pluronics), chitosan, gelatin, cellulose derivatives, glycol chitin, poly(N-isopropylacrylamide) (PNIPAAm), PEG-PLGA-PEG, poly(D, L-lactide)-poly(ethyleneglycol)-poly(D,L-lactide) (PDLLA-PEG-PDLLA), and combinations thereof. In some embodiments, the hydrogel comprises chitosan and glycol chitosan. In some embodiments, the hydrogel comprises glycol chitin. In some embodiments, the hydrogel is an amphiphilic block copolymer comprising at least on hydrophobic polymer block and at least one hydrophilic polymer block. In some embodiments, the amphiphilic block copolymer is PEG-PLGA-PEG or PDLLA-PEG-PDLLA.

### Additives

In some embodiments, the nanoparticles further include thermal stabilizers. Examples of useful thermal stabilizers include phenolic antioxidants such as butylated hydroxytoluene (BHT), 2-t-butylhydroquinone, and 2-t-butylhydroxyanisole.

In some embodiments, the nanoparticles further include one or more surfactants. In some embodiments, the surfactant may include cationic, amphoteric, or non-ionic surfactants, or a combination thereof. In some embodiments, the surfactants comprise anionic surfactants selected from the group consisting of fatty acid salts, bile salts, phospholipids, carnitines, ether carboxylates, succinylated monoglycerides, mono/diacetylated tartaric acid esters of mono- and diglycerides, citric acid esters of mono- and diglycerides, sodium oleate, sodium lauryl sulfate, sodium lauryl sarcosinate, sodium dioctyl sulfosuccinate (SDS), sodium cholate, sodium taurocholate, lauroyl carnitine, palmitoyl carnitine, myristoyl carnitine, lactylic esters of fatty acids, and combinations thereof. In some embodiments, anionic surfactants include di-(2-ethylhexyl) sodium sulfosuccinate. In some embodiments, the surfactants are non-ionic surfactants selected from the group consisting of propylene glycol fatty acid esters, mixtures of propylene glycol fatty acid esters and glycerol fatty acid esters, triglycerides, sterol and sterol derivatives, sorbitan fatty acid esters and polyethylene glycol sorbitan fatty acid esters, sugar esters, polyethylene glycol alkyl ethers and polyethylene glycol alkyl phenol ethers, polyoxyethylene-polyoxypropylene block copolymers, lower alcohol fatty acid esters, and combinations thereof. In some embodiments, the surfactants may comprise fatty acids. Examples of fatty acids include caprylic acid, undecylic acid, lauric acid, tridecylic acid, myristic acid, palmitic acid, stearic acid, or oleic acid. In some embodiments, the surfactants comprise amphoteric surfactants including (1) substances classified as simple, conjugated and derived proteins such as the albumins, gelatins, and glycoproteins, and (2) substances contained within the phospholipid classification, for example lecithin. The amine salts and the quaternary ammonium salts within the cationic group also comprise useful surfactants.

In some embodiments, the surfactant comprises a hydrophilic amphiphilic surfactant polyoxyethylene (20) sorbitan monolaurate (TWEEN® 20) or polyvinyl alcohol that improves the distribution of IR absorbing material in the polymeric carrier. In some embodiments, the surfactant comprises an amphiphilic surfactant if the IR absorbing material is hydrophilic and the polymeric carrier is hydrophobic. In some embodiments, the surfactant is an anionic surfactant sodium bis(tridecyl) sulfosuccinate (Aerosol® TR-70). In some embodiments, the surfactant is sodium bis(tridecyl) sulfosuccinate, or sodium dodecyl sulfate (SDS).

In an embodiment, the surfactant is selected from the group consisting of polysorbate, polyvinyl alcohol, methyl cellulose, gelatin, albumin, poloxamer, ethyl cellulose, crosslinked polyacrylic acid polymer, tocopheryl polyethylene glycol succinate (TPGS), sodium cholate, lipids, stearic acid, and combinations thereof.

### Pharmaceutical Compositions

In some embodiments, the disclosure provides pharmaceutical compositions comprising a BTK inhibitor compound for treating an ophthalmic condition, wherein the ophthalmic condition includes: ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The pharmaceutical composition is administered in an amount effective to treat the ophthalmic condition.

In some embodiments, the disclosure provides pharmaceutical compositions comprising a BTK inhibitor compound for reducing inflammation in an eye of a human in need thereof. The pharmaceutical composition is administered in an amount effective to reduce inflammation in the eye of the human.

In some embodiments, the disclosure provides pharmaceutical compositions comprising an BTK inhibitor compound for reducing an immune response in a human in need thereof having an ophthalmic condition comprising the step of administering to the human a BTK inhibitor compound. The immune response includes an innate immune response, an adaptive immune response, or both. The ophthalmic condition includes ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis. The pharmaceutical composition is administered in an amount effective to reduce the immune response in the human having an ophthalmic condition.

The pharmaceutical compositions are typically formulated to provide a therapeutically effective amount of an BTK inhibitor or a pharmaceutically acceptable salt thereof. Where desired, the pharmaceutical compositions contain a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants. Where desired, other ingredients in addition to a BTK inhibitor or a pharmaceutically acceptable salt thereof may be mixed into a preparation or both components may be formulated into separate preparations for use in combination separately or at the same time.

In selected embodiments, the concentration of a BTK inhibitor or a pharmaceutically acceptable salt thereof provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v.

In selected embodiments, the concentration of a BTK inhibitor or a pharmaceutically acceptable salt thereof provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v.

In selected embodiments, the concentration of a BTK inhibitor or a pharmaceutically acceptable salt thereof is independently in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12% or approximately 1% to approximately 10% w/w, w/v or v/v.

In selected embodiments, the concentration of a BTK inhibitor or a pharmaceutically acceptable salt thereof is independently in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In selected embodiments, the amount of a BTK inhibitor or a pharmaceutically acceptable salt thereof is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g.

In selected embodiments, the amount of a BTK inhibitor or a pharmaceutically acceptable salt thereof is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g or 10 g.

A BTK inhibitor or a pharmaceutically acceptable salt thereof are effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

The pharmaceutical composition may be provided in various forms, including in the form of tablets, gelatin capsules, dragées, syrups, suspensions, solutions, powders, granules, emulsions, or suspensions of microspheres or nanospheres or of lipid or polymeric vesicles for controlled release.

In an embodiment, the BTK inhibitor compound is in a dosage form selected from a solution, suspension, emulsion, microemulsion, ointment, gel, hydrogel, drug delivery device, tablet, or capsule. In an embodiment, the drug delivery device is an ocular insert for sustained release of the BTK inhibitor compound. Ocular inserts include solid and semi-solid devices, usually made of polymeric materials, into which the BTK inhibitor is loaded. In an embodiment, the dosage form is a sustained release form, an extended release form, a controlled release form, or a combination thereof. In an embodiment, the sustained release, extended release, or controlled release dosage form comprises a pegylated BTK inhibitor.

In an embodiment, the BTK inhibitor compound is administered as particles that self-aggregate into a depot upon administration. In an embodiment, the particles further comprise a polymer. In an embodiment, the polymer is selected from the group consisting of chitosan, gelatin, sodium alginate, albumin, poly-L-lactide (PLLA), poly(lactic acid) (PLA), poly(glycolic acid)(PGA), poly(lactic co-glycolic acid) (PLGA), polycaprolactone, poly(lactide co-caprolactone), poly(methyl methacrylates), poloxamer, poly(ethylene glycol) (PEG), PEG-PLLA, PEG-PLGA, poly(methyl vinyl ether/maleic anhydride), cellulose acetate phthalate, and combinations thereof. In an embodiment, the polymer is poly(lactic co-glycolic acid) (PLGA), PEG-PLGA, or a combination thereof. In an embodiment, the polymer is poly(lactic co-glycolic acid) (PLGA). In an embodiment, the polymer is PEG-PLGA. In an embodiment, the polymer is a combination of poly(lactic co-glycolic acid) (PLGA) and PEG-PLGA. In an embodiment, the particles are administered by intravitreal injection.

Described below are non-limiting exemplary pharmaceutical compositions and methods for preparing the same.

### Pharmaceutical Compositions for Topical Administration

Pharmaceutical compositions for the topical ophthalmic administration of this disclosure may be formulated in conventional ophthalmologically compatible vehicles, such as, for example, an ointment, cream, suspension, lotion, powder, solution, paste, gel, hydrogel, spray, aerosol or oil.

The formulation may be one of many topical formulation types containing water as the major ingredient, including solutions, gels, hydrogel, creams, sprays and foams. In an embodiment, the formulation may be in the form of an aqueous gel. Accordingly, the formulation of the disclosure for the topical ophthalmic administration may contain a gelling or thickening agent. Any gelling agent that is water-dispersible is suitable for use in the composition of the disclosure. One preferred gelling agent is hydroxypropylcellulose, such as that sold under the tradename KLUCEL® (Hercules Incorporated). Another preferred gelling agent is hydroxyethylcellulose, such as that sold under the tradename NATROSOL® (Hercules Incorporated). Other suitable gelling agents include carboxyvinyl polymers, also known as carbomers, such as are sold under the tradename CARBOPOL® 934, 940, 941, 980, and 981 (B.F. Goodrich Co.), ETD 2020™, and ULTREZ® (Noveon Inc.). Additional suitable gelling agents are polyvinyl alcohol, polyethylene oxides, propylene glycol alginates, methylcellulose, hydroxypropylmethylcellulose and natural polymeric gums such as xanthan, and carrageenan. The concentration of gelling agent in the composition may be varied depending on several factors, including the desired degree of stabilization of the suspension and desired viscosity of the gel composition.

If desired, the formulation of the disclosure may further include additional pharmaceutically acceptable excipients typically used in formulations and known to those skilled in the art. Such excipients include, for example, humectants, emollients, pH stabilizing agents, preservatives, chelating agents, and anti-oxidants.

The formulation of the disclosure for the topical ophthalmic administration may be made by any means by which the components of the disclosure are combined to provide a pharmaceutical formulation. For example, a suspension of benzoyl peroxide may be made by combining water, the water-miscible organic solvent, and benzoyl peroxide. Preferably, the combination is mixed, such as by stirring, sonicating, milling, and/or shaking, to produce a uniform suspension of benzoyl peroxide particles in the water and organic solvent. Additional ingredients, such as a gelling agent and other excipients, may be added either before or after the uniform suspension is obtained.

Gels comprising polymers can swell in water and then interact in such a way as to thicken the water and increase viscosity. Polymers may interact physically, by chain entanglement, or by ionic or hydrophobic/hydrophilic interactions. In each case, the polymers form a matrix that increases the viscosity of the water and allows for (1) physical stabilization and prevention of migration of suspended BTK inhibitor, (2) maintenance of product homogeneity throughout the shelf life, (3) clean, no drip, no mess transfer of the product from the primary package to the skin surface and (4) easy spreading and acceptable aesthetics.

In an embodiment, the composition for the topical ophthalmic administration comprises matrix builder, such as high molecular weight polyvinylpyrrolidones (e.g., Kollidon® 90F), thicking polymers and biopolymers; poloxamers, emulsifiers, stably suspending oils in gels and solubilizers. The composition for the topical ophthalmic administration may have sensory modifiers such as isopropyl myristate. The solubility in an aqueous matrix can be enhanced by the use of water miscible solvents like propylene glycol, polyethylene glycols, triacetin, poloxamers, and low molecular weight polyvinylpyrrolidone.

In an embodiment, the composition for the topical ophthalmic administration comprises the BTK inhibitor suspended in a hydrogel. A hydrogel is a colloidal gel formed as a dispersion in water or other aqueous medium. Thus a hydrogel is formed upon formation of a colloid in which a dispersed phase (the polymer) has combined with a continuous phase (i.e. water) to produce a viscous jellylike product; for example, coagulated silicic acid. A hydrogel is a three-dimensional network of hydrophilic polymer chains that are cross-linked through either chemical or physical bonding. Because of the hydrophilic nature of the polymer chains, hydrogels absorb water and swell (unless they have already absorbed their maximum amount of water). The swelling process is the same as the dissolution of non-cross-linked hydrophilic polymers. By definition, water constitutes at least 10% of the total weight (or volume) of a hydrogel.

Examples of hydrogels include synthetic polymers such as polyhydroxy ethyl methacrylate, and chemically or physically cross-linked polyvinyl alcohol, polyacrylamide, poly(N-vinyl pyrolidone), polyethylene oxide, and hydrolysed polyacrylonitrile. Examples of hydrogels which are organic polymers include covalent or ionically cross-linked polysaccharide-based hydrogels such as the polyvalent metal salts of alginate, pectin, carboxymethyl cellulose, heparin, hyaluronate and hydrogels from chitin, chitosan, pullulan, gellan and xanthan. The preferred hydrogels includes a cellulose compound (i.e. hydroxypropylmethylcellulose [HPMC]) and/or a high molecular weight hyaluronic acid (HA).

The pharmaceutical compositions according to the disclosure the topical ophthalmic administration may also contain inert additives or combinations of these additives, such as wetting agents; mucoadhesive agents; flavor enhancers; preservatives such as para-hydroxybenzoic acid esters; stabilizers; moisture regulators; pH regulators; osmotic pressure modifiers; emulsifiers; UV-A and UV-B screening agents; and antioxidants, such as α-tocopherol, butylhydroxyanisole or butylhydroxytoluene, superoxide dismutase, ubiquinol or certain metal chelating agents.

The formulations for the topical ophthalmic administration, after sterilization, may be packaged, stored and used directly. In an exemplary embodiment, the formulations are in drop form in the manner typically used to apply eye drops. The normal squeeze-type liquid drop application devices are perfectly suited for use in applying the ophthalmic formulations of the disclosure. In an exemplary embodiment, the formulations are conveniently administered by dropwise addition of the formulations into the affected eye(s) of the user.

The formulations of the present disclosure for the topical ophthalmic administration containing preservatives are especially advantageous for use in multi-dose containers. Multi-dose containers, as used herein, refer to containers which allow two or more separate applications of the ophthalmic formulation present within the container. Such containers are resealable—i.e., the container cap may be removed for a first application, and then the cap may be replaced onto the container, thereby providing a substantially liquid impermeable seal again. In various exemplary embodiments, an antimicrobial preservative is present in an amount sufficient to reduce microbial concentrations for a period of about 12 hours to about 1 month, such as about 12 hours to about 3 weeks, such as about 12 hours to about 2 weeks, such as about 12 hours to about 1 week, such as about 12 hours to about 3 days, such as about 12 hours to about 48 hours, such as about 12 hours to about 24 hours.

In an exemplary embodiment, those formulations containing no preservative are packaged in a unit dose container—i.e., where only a single dose can be provided by a given container. Such preservative-free compositions are subject to uncontrolled microbial growth once the consumer initially breaks the container seal. Accordingly, the consumer is instructed to dispose of the container after the first dose. An appropriate unit-dose system such as blow-fill-seal unit dose preservative-free packaging system is typically used for the preservative-free formulations.

The concentration of the BTK inhibitor or pharmaceutically acceptable salts thereof for topical ophthalmic administration is typically about 0.01% to about 10.0% by weight, about 0.02% to about 9.0% by weight, about 0.03% to about 8.0% by weight, about 0.04% to about 7.0% by weight, about 0.05% to about 8.0% by weight, about 0.06% to about 7.0% by weight, about 0.07% to about 6.0% by weight, about 0.08% to about 5.0% by weight, about 0.09% to about 4.0% by weight, about 0.1% to about 3.0% by weight, about 0.2% to about 2.0% by weight, about 0.3% to about 1.0% by weight, about 0.4% to about 5.0% by weight, or about 0.5% to about 5.0% by weight.

The concentration of the BTK inhibitor or pharmaceutically acceptable salts thereof for topical ophthalmic administration is typically about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.15% by weight, about 0.20% by weight, about 0.25% by weight, about 0.30% by weight, about 0.35% by weight, about 0.40% by weight, about 0.45% by weight, about 0.50% by weight, about 0.55% by weight, about 0.6% by weight, about 0.65% by weight, about 0.7% by weight, about 0.75% by weight, about 0.8% by weight, about 0.85% by weight, about 0.9% by weight, about 0.95% by weight, about 1% by weight, about 2% by weight, about 3% by weight, about 4% by weight, about 5% by weight, about 6% by weight, about 7% by weight, about 8% by weight, about 9% by weight, or about 10% by weight.

In various exemplary embodiments, the BTK inhibitor or pharmaceutically acceptable salts thereof is employed at a concentration of about 0.1 to about 10% w/v, such as about 0.1 to about 4.5% w/v, such as about 0.1 to about 4.0% w/v, such as about 0.1 to about 3.5% w/v, such as about 0.1 to about 3.0% w/v, such as about 0.1 to about 2.5% w/v, such as about 0.1 to about 2.0% w/v, such as about 0.1 to about 1.5% w/v, such as about 0.1 to about 1.0% w/v, such as about 0.1 to about 0.8% w/v, such as about 0.1 to about 0.7% w/v, such as about 0.1 to about 0.6% w/v, such as about 0.1 to about 0.5% w/v, such as about 0.1 to about 0.4% w/v, such as about 0.1 to about 0.3% w/v, such as about 0.1 to about 0.2% w/v.

Optionally, the formulations for the topical ophthalmic administration contain a tonicity modifier.

In an exemplary embodiment, the tonicity modifier is non-ionic. The tonicity modifier may be selected from, but is not limited to, mannitol, sorbitol, dextrose, sucrose, urea, glycerol, polyethylene glycol and any mixtures thereof. In an exemplary embodiment, the tonicity modifier is present in amount sufficient to generate a tonicity of about 250 to about 350 milliosmoles per kilogram (mOsmol/kg), such as about 265 to about 325 mOsmol/kg, such as about 280 to about 310 mOsmol/kg, such as about 295 to about 315 mOsmol/kg.

The formulation for the topical ophthalmic administration may also contain, an ionic salt, selected from, but not limited to, alkali metal halides (such as, for example, NaCl, KCl, NaBr, etc.), in an amount ranging from about 0.3% to about 1% weight percent or sufficient to approximate the salt concentration and/or tonicity of the human tear fluid. Selected salts from this group may also be referred to as ionic tonicity modifiers.

Where a preservative is used in the formulations for the topical ophthalmic administration, an antimicrobial is present in an amount sufficient to generate a microbial barrier to maintain or reduce microbial concentrations for a period of about 12 hours to about 1 month, such as about 12 hours to about 3 weeks, such as about 12 hours to about 2 weeks, such as about 12 hours to about 1 week, such as about 12 hours to about 3 days, such as about 12 hours to about 48 hours, such as about 12 hours to about 24 hours. Suitable preservatives include, but are not limited to, benzalkonium chloride, benzyl alcohol, sorbic acid, chlorobutanol, cetrimonium, methylparaben, propylparaben, polyamino propyl biguanide, phenylethyl alcohol, chlorhexidine, chlorhexidine digluconate, chloroquat, stabilized oxychloro complex or any combination thereof.

Buffering agents that can be used in the formulations for the topical ophthalmic administration include, but are not limited to, buffers prepared from sodium, potassium bicarbonate, phosphate, acetate, citrate, borate salts and/or phosphoric acid, acetic acid, citric acid or boric acid. In an exemplary embodiment, the buffer is sodium dihydrogen phosphate or disodium phosphate or boric acid/sodium borate. The buffers of the disclosure should be present in an amount sufficient to produce and maintain a formulation pH of about 5.0 to about 8.0, such as about 5.5 to about 7.7, such as about 6.0 to about 7.5, such as about 6.3 to about 7.5, such as about 6.7 to 7.5, such as about 6.7 to about 7.1, and including a pH of about 5.7, about 5.9, about 6.1, about 6.3, about 6.5, about 6.7, about 6.9, about 7.1, about 7.3, about 7.5, about 7.7 or about 7.9.

A surfactant may also be added to the compositions for the topical ophthalmic administration. In an exemplary embodiment, the surfactant is present at a concentration range of about 0.001% to about 0.3%, such as about 0.005% to about 0.2%, such as about 0.01% to about 0.1%, such as about 0.05% to about 0.1% to provide enhanced wetting characteristics to the formulation. The surfactant may include, but is not limited to, poloxamers, polysorbate 80, polysorbate 20, tyloxapol, polyoxyethylene, Brij 35, Brij 58, Brij 78, Aptet 100, G 1045, Spans 20, 40 and 85, Tweens 20, 40, 80 or 81, sodium lauroyl sarcosinate, lauroyl-L-glutamic acid triethanolamine, sodium myristyl sarcosinate and sodium lauryl sulfate, polyoxyethylene sorbitan fatty acid esters, polyoxyethylene hydrogenated castor oil, polyethylene glycol fatty acid esters (e.g., polyoxyl stearate), polyoxyethylene polyoxypropylene alkyl ethers, polyoxyalkylene alkyl phenyl ethers, polyglycerol fatty acids esters (e.g., decaglycerol monolaurate), glycerol fatty acid esters, sorbitan fatty acid esters, and polyoxyethylene polyoxypropylene glycol (poloxamer), polyoxyl stearate 40, and/or any combination thereof.

A stabilizer can also be added to the formulations for the topical ophthalmic administration. Suitable stabilizers include, but are not limited to, sodium metabisulfite, sodium bisulfate, acetylcysteine, ascorbic acid, sodium thiosulfate, alpha-tocopherol, carnosine, retinyl palmitate, salts of ethylenediaminetetraacetic acid (EDTA) (such as, for example, the disodium, tetrasodium, calcium or calcium sodium edetate salts), or any combination thereof.

The mucoadhesive agent, when present in the described formulations, increases corneal contact time, enhances bioavailability and/or produces a lubricating effect, and includes, but is not limited to acrylic acid polymers, methylcellulose, ethylcellulose, Povidone K-30, hydroxypropyl methylcellulose, hydroxyethylcellulose, Carbopol® polymers (such as, for example, Carbopol® 674, 676, 690, 980 NF, EZ-2, EZ-3, EZ-4, Aqua 30 and Novethix™ L-10), hydroxypropyl cellulose, polyvinyl alcohol, gelatin, sodium chondroitin sulfate, or any combination thereof.

In an embodiment, after administration onto the surface of the eye, the composition enters the conjunctiva and anterior sclera and into the corneal layer. When present, the mucoadhesive agent appears to increase residence time in the cornea so that the drug may diffuse slowly over time to the posterior sclera, resulting in delivery of sustained concentrations of the BTK inhibitor or pharmaceutically acceptable salts thereof in the posterior sclera. The mucoadhesive agent accomplishes this objective by retarding the loss of the drug through, for example, drainage from the nasolacrimal duct due to lachrymation and tear turnover. The mucoadhesive agent also typically possesses viscosity enhancing properties that may result in a desirable soothing or lubricating effect. The penetration enhancer agent which is optionally added to the formulation enhances penetration of the formulation into the corneal epithelial layers, further enhancing the residence time of the BTK inhibitor or pharmaceutically acceptable salts thereof in the eye. The stabilizing agent may act as an antioxidant or otherwise retard the chemical degradation of the BTK inhibitor formulation. The buffering agent buffers the formulation to a comfortable near-neutral pH compatible with ocular administration. The tonicity modifier in the formulation produces the appropriate osmolality of the ophthalmic formulation.

The penetration enhancer optionally present in the described formulations for the topical ophthalmic administration includes, but is not limited to, laurocapram (azone), bile acids and their alkali metal salts, including chenodeoxycholic acid, cholic acid, taurocholic acid, taurodeoxycholic acid, tauroursodeoxycholic acid or ursodeoxycholic acid, glycocholate, n-dodecyl-β-D-maltoside, sucrose dodecanoate, octyl maltoside, decyl maltoside, tridecyl maltoside, tetradecyl maltoside, hexamethylene lauramide, hexamethylene octanamide, glycerol monolaurate, PGML (polyethylene glycol monolaurate), dimethyl sulfoxide, methylsulfonylmethane, sodium fusidate, saponins, cyclodextrins (CDs) or any combination thereof.

In addition, a solubilizing or resuspension agent may also be added to the formulations for the topical ophthalmic administration. Suitable solubilizing or resuspension agents include, but are not limited to, cyclodextrins (CDs), such as hydroxypropyl γ-cyclodextrin (Cavasol®), sulfobutyl ether 4 β-cyclodextrin (Captisol®), and hydroxypropyl β-cyclodextrin (Kleptose®) (such as 2-hydroxypropyl β-cyclodextrin), Polysorbate 80 (Tween80®) or hyaluronic acid or hyaluronate salts. The cyclodextrins in particular may also exhibit penetration enhancing properties, although in other instances, cyclodextrins are known to retard the uptake of steroidal compounds (such as hydrocortisone) into ocular tissues. Masson, Proc. of the 9^{th} Intl. Symposium on Cyclodextrins, Kluwer Academic Publishers (1999), 363-369; Loftsson, Acta Ophthalmologica Scandinavica (2003), 144-150; International Journal of Pharmaceutics 156 (1997), 201-209.

An exemplary listing of typical carriers, stabilizers and adjuvants known to those of skill in the art that may be useful in the ophthalmic compositions described herein may be found in Gennaro (2005) Remington: The Science and Practice of Pharmacy, Mack Publishing, 21^{st} ed.

### Pharmaceutical Compositions for Injection

Pharmaceutical compositions for injection may be formulated with a pharmaceutical excipient suitable for injection. Components and amounts of agents in the compositions are as described herein.

Aqueous solutions in saline are also conventionally used for injection. Ethanol, glycerol, propylene glycol and liquid polyethylene glycol (and suitable mixtures thereof), and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating a BTK inhibitor or a pharmaceutically acceptable salt thereof in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Administration of a BTK inhibitor or a pharmaceutically acceptable salt thereof or pharmaceutical composition of these compounds can be effected by any method that enables delivery of the compounds to the site of action. These methods include oral routes, intraduodenal routes, parenteral injection (including intravenous, intra-arterial, subcutaneous, intramuscular, intravascular, intraperitoneal or infusion), topical (e.g., transdermal application), rectal administration, via local delivery by catheter or stent or through inhalation. The compounds can also be administered intraadiposally or intrathecally.

In an embodiment, the pharmaceutical composition comprising a BTK inhibitor or a pharmaceutically acceptable salt thereof is administered by intravitreal injection.

In an embodiment, the pharmaceutical composition comprising a BTK inhibitor or a pharmaceutically acceptable salt thereof is administered by intraocular injection.

Exemplary parenteral administration forms include solutions or suspensions of active compound in sterile aqueous solutions, for example, aqueous propylene glycol or dextrose solutions. Such dosage forms can be suitably buffered, if desired.

The disclosure also provides kits. The kits include a pharmaceutical composition comprising a BTK inhibitor or a pharmaceutically acceptable salt thereof, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer. In an embodiment, the disclosure provides a kit of a pharmaceutical composition comprising a BTK inhibitor or a pharmaceutically acceptable salt thereof for use in the treatment of an ophthalmic condition described herein.

### Dosages and Dosing Regimens

The amount of a BTK inhibitor or a pharmaceutically acceptable salt thereof administered will be dependent on the human being treated, the severity of the disorder or condition, the rate of administration, the disposition of the compounds and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day.

In some embodiments, a BTK inhibitor or a pharmaceutically acceptable salt thereof is administered in a single dose. Typically, such administration will be by injection—e.g., intravenous injection or intravitreal injection, in order to introduce the agents quickly. However, other routes may be used as appropriate. A single dose of a BTK inhibitor or a pharmaceutically acceptable salt thereof may also be used for treatment of an acute condition.

In some embodiments, a BTK inhibitor or a pharmaceutically acceptable salt thereof is administered in multiple doses for treating an ophthalmic condition. In an embodiment, a BTK inhibitor or a pharmaceutically acceptable salt thereof is administered in multiple doses. In an embodiment, a BTK inhibitor or a pharmaceutically acceptable salt thereof is administered in multiple doses by injection—e.g., intravenous injection or intravitreal injection. In an embodiment, dosing may be once, twice, three times, four times, five times, six times, or more than six times per day. In an embodiment, dosing may be selected from the group consisting of once a day, twice a day, three times a day, four times a day, five times a day, six times a day, once every other day, once weekly, twice weekly, three times weekly, four times weekly, biweekly, and monthly. In other embodiments, a BTK inhibitor or a pharmaceutically acceptable salt thereof is administered about once per day to about six times per day. In some embodiments a BTK inhibitor or a pharmaceutically acceptable salt thereof is administered once daily, while in other embodiments a BTK inhibitor or a pharmaceutically acceptable salt thereof is administered twice daily, and in other embodiments a BTK inhibitor or a pharmaceutically acceptable salt thereof is administered three times daily. In some embodiments a BTK inhibitor or a pharmaceutically acceptable salt thereof is administered three times a week, including every Monday, Wednesday, and Friday.

In some embodiments, a pharmaceutical composition comprising a BTK inhibitor is administered by intravitreal or intraocular injection to a human subject monthly, bi-monthly, once every three months, quarterly, once every five months, once every six months, or yearly. In some embodiments, the pharmaceutical composition comprising a BTK inhibitor is administered by intravitreal or intraocular injection to a human subject monthly for two, three, four, or five months followed by bi-monthly administration.

In some embodiments, a pharmaceutical composition comprising a BTK inhibitor is administered topically to a human subject once a day, twice a day, three times a day, once every other day, weekly, twice weekly, three times weekly, four times weekly, biweekly, or monthly.

Administration of a BTK inhibitor or a pharmaceutically acceptable salt thereof may continue as long as necessary. In some embodiments, a BTK inhibitor or a pharmaceutically acceptable salt thereof is administered for more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. In some embodiments, a BTK inhibitor or a pharmaceutically acceptable salt thereof is administered for less than 28, 14, 7, 6, 5, 4, 3, 2, or 1 day. In some embodiments, a BTK inhibitor or a pharmaceutically acceptable salt thereof is administered for about 14 days, about 21 days, about 28 days, about 35 days, about 42 days, about 49 days, or about 56 days. In some embodiments, a BTK inhibitor or a pharmaceutically acceptable salt thereof is administered chronically on an ongoing basis—e.g., for the treatment of chronic effects. In another embodiment the administration of a BTK inhibitor or a pharmaceutically acceptable salt thereof continues for less than about 7 days. In yet another embodiment the administration continues for more than about 6, 10, 14, 28 days, two months, three months, four months, five months, six months, seven months, eight months, nine months, ten months, eleven months or one year. In some embodiments, the administration continues for more than about one year, two years, three years, four years, or five years. In some embodiments, continuous dosing is achieved and maintained as long as necessary.

In some embodiments, an effective dosage of a BTK inhibitor or a pharmaceutically acceptable salt thereof is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of a BTK inhibitor or a pharmaceutically acceptable salt thereof is about 15 mg, about 25 mg, about 30 mg, about 50 mg, about 50 mg, about 75 mg, about 90 mg, about 100 mg, about 120 mg, about 125 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, about 400 mg, about 425 mg, about 450 mg, about 475 mg, about 480 mg, or about 500 mg. In some embodiments, an effective dosage of a BTK inhibitor or a pharmaceutically acceptable salt thereof is 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg.

In some embodiments, an effective dosage of a BTK inhibitor or a pharmaceutically acceptable salt thereof is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of a BTK inhibitor or a pharmaceutically acceptable salt thereof is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, a BTK inhibitor or a pharmaceutically acceptable salt thereof is administered at a dosage of 10 to 500 mg BID, including a dosage of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg BID.

In some embodiments, a BTK inhibitor or a pharmaceutically acceptable salt thereof is administered at a dosage of 10 to 500 mg QD, including a dosage of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg QD.

An effective amount of a BTK inhibitor or a pharmaceutically acceptable salt thereof may be administered in either single or multiple doses by any of the accepted modes of administration of agents having similar utilities, including buccal, sublingual, and transdermal routes, by intra-arterial injection, intravenously, parenterally, intramuscularly, subcutaneously or orally.

In certain embodiments, the route of delivery used is intraocular injection, direct injection into a given compartment of the eye, such as the vitreous, the cornea, or the retina, application of a patch on the eye, direct application of an ointment, spray, or droppable liquid to the eye, or intraocular implant. In an embodiment, the route of delivery is intravitreal injection.

In some embodiments, the route of delivery used is topical administration to an eye of a human in need thereof, intraocular injection to an eye of a human in need thereof, intravitreal injection to an eye of a human in need thereof, periocular administration to a human in need thereof, oral administration to a human in need thereof, intravenous injection to a human in need thereof, or a combination thereof.

In some embodiments, the pharmaceutical composition comprising a BTK inhibitor or a pharmaceutically acceptable salt thereof is administered by injection at a dosage of about 0.001 mg/ml, about 0.005 mg/ml, about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4.0 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, about 5.0 mg/ml, about 5.1 mg/ml, about 5.2 mg/ml, about 5.3 mg/ml, about 5.4 mg/ml, about 5.5 mg/ml, about 5.6 mg/ml, about 5.7 mg/ml, about 5.8 mg/ml, about 5.9 mg/ml, about 6.0 mg/ml, about 6.1 mg/ml, about 6.2 mg/ml, about 6.3 mg/ml, about 6.4 mg/ml, about 6.5 mg/ml, about 6.6 mg/ml, about 6.7 mg/ml, about 6.8 mg/ml, about 6.9 mg/ml, about 7.0 mg/ml, about 7.1 mg/ml, about 7.2 mg/ml, about 7.3 mg/ml, about 7.4 mg/ml, about 7.5 mg/ml, about 7.6 mg/ml, about 7.7 mg/ml, about 7.8 mg/ml, about 7.9 mg/ml, about 8.0 mg/ml, about 8.1 mg/ml, about 8.2 mg/ml, about 8.3 mg/ml, about 8.4 mg/ml, about 8.5 mg/ml, about 8.6 mg/ml, about 8.7 mg/ml, about 8.8 mg/ml, about 8.9 mg/ml, about 9 mg/ml, about 9.1 mg/ml, about 9.2 mg/ml, about 9.3 mg/ml, about 9.4 mg/ml, about 9.5 mg/ml, about 9.6 mg/ml, about 9.7 mg/ml, about 9.8 mg/ml, about 9.9 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, or about 50 mg/ml.

In some embodiments, the pharmaceutical composition comprising a BTK inhibitor or a pharmaceutically acceptable salt thereof is administered by injection at a volume of about 0.01 ml, about 0.02 ml, about 0.03 ml, about 0.04 ml, about 0.05 ml, about 0.06 ml, about 0.07 ml, about 0.08 ml, about 0.09 ml, about 0.1 ml, about 0.15 ml, about 0.2 ml, about 0.25 ml, about 0.30 ml, about 0.35 ml, about 0.40 ml, about 0.45 ml, about 0.5 ml, about 0.55 ml, about 0.60 ml, about 0.65 ml, about 0.70 ml, about 0.75 ml, about 0.80 ml, about 0.85 ml, about 0.90 ml, about 0.95 ml, about 1.0 ml, about 1.1 ml, about 1.2 ml, about 1.3 ml, about 1.4 ml, about 1.5 ml, about 1.6 ml, about 1.7 ml, about 1.8 ml, about 1.9 ml, about 2.0 ml, about 2.5 ml, about 3.0 ml, about 3.5 ml, about 4.0 ml, about 4.5 ml, about 5.0 ml, about 5.5 ml, about 6.0 ml, about 6.5 ml, about 7.0 ml, about 7.5 ml, about 8.0 ml, about 8.5 ml, about 9.0 ml, about 9.5 ml, about 10.0 ml, about 15.0 ml, about 20.0 ml, about 25.0 ml, about 30.0 ml, about 35.0 ml, about 40.0 ml, about 45.0 ml, or about 50.0 ml.

In some embodiments, the pharmaceutical composition comprising a BTK inhibitor or a pharmaceutically acceptable salt thereof is administered by intravitreal or intraocular injection at a volume of about 0.001 ml, about 0.005 ml, about 0.010 ml, about 0.015 ml, about 0.020 ml, about 0.025 ml, about 0.030 ml, about 0.035 ml, about 0.040 ml, about 0.045 ml, about 0.05 ml, about 0.055 ml, about 0.06 ml, about 0.065 ml, about 0.07 ml, about 0.075 ml, about 0.08 ml, about 0.085 ml, about 0.09 ml, about 0.095 ml, or about 0.1 ml.

In some embodiments, a BTK inhibitor or a pharmaceutically acceptable salt thereof is administered to a subject intermittently, known as intermittent administration. By “intermittent administration”, it is meant a period of administration of a therapeutically effective dose of a BTK inhibitor or a pharmaceutically acceptable salt thereof, followed by a time period of discontinuance, which is then followed by another administration period and so on. In each administration period, the dosing frequency can be independently select from three times daily, twice daily, daily, once weekly, twice weekly, three times weekly, four times weekly, five times weekly, or six times weekly for topical administration to the eye or monthly for intravitreal or intraocular injection to the eye.

By “period of discontinuance” or “discontinuance period” or “rest period”, it is meant to the length of time when discontinuing of the administration of a BTK inhibitor or a pharmaceutically acceptable salt thereof. The time period of discontinuance may be longer or shorter than the administration period or the same as the administration period. During the discontinuance period, other therapeutic agents other than a BTK inhibitor or a pharmaceutically acceptable salt thereof may be administered.

In an embodiment, a pharmaceutical composition comprising a BTK inhibitor or a pharmaceutically acceptable salt thereof is administered to a human subject in need thereof by intravitreal or intraocular injection for treating an ophthalmic condition for a first administration period, then followed by a discontinuance period, then followed by a second administration period, and so on, wherein the ophthalmic condition includes: ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis.

In an embodiment, a pharmaceutical composition comprising a BTK inhibitor or a pharmaceutically acceptable salt thereof is administered topically to a human subject in need thereof for treating an ophthalmic condition for a first administration period, then followed by a discontinuance period, then followed by a second administration period, and so on, wherein the ophthalmic condition includes: ocular inflammation, dry eye disease (including aqueous-deficient dry eye disease, hyperevaporative dry eye disease, and mixed aqueous-deficient and hyperevaporative dry eye disease), uveitis (including infectious uveitis, non-infectious uveitis, anterior uveitis, intermediate uveitis, posterior uveitis, and panuveitis), post-operative ocular inflammation, corneal transplantation, ocular graft-versus-host disease (GVHD), allergy, allergic conjunctivitis, non-allergic conjunctivitis, and infectious conjunctivitis.

For topical administration to the eye, the first administration period, the second administration period, and the discontinuance period are independently selected from the group consisting of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, one month, five weeks, six weeks, seven weeks, two months, nine weeks, ten weeks, elven weeks, three months, thirteen weeks, fourteen weeks, fifteen weeks, four months, and more days, in which a BTK inhibitor or a pharmaceutically acceptable salt thereof is administered to a subject three times daily, twice daily, daily, once weekly, twice weekly, three times weekly, four times weekly, five times weekly, six times weekly or monthly. In an embodiment, the first administration period is the same length as the second administration period. In an embodiment, the first administration period is shorter than the second administration period. In an embodiment, the first administration period is longer than the second administration period. In an embodiment, the first administration period and the second administration period are about one week, in which a BTK inhibitor or a pharmaceutically acceptable salt thereof is administered to a subject daily; and the discontinuance period is about two weeks. In an embodiment, the first administration period and the second administration period are about three weeks, in which a BTK inhibitor or a pharmaceutically acceptable salt thereof is administered to a subject daily; and the discontinuance period is about two weeks. In an embodiment, the first administration period and the second administration period are about three weeks, in which a BTK inhibitor or a pharmaceutically acceptable salt thereof is administered to a subject weekly; and the discontinuance period is about two weeks. In an embodiment, the first administration period and the second administration period are about four weeks, in which a BTK inhibitor or a pharmaceutically acceptable salt thereof is administered to a subject daily; and the discontinuance period is about two weeks. In an embodiment, the first administration period and the second administration period are about four weeks, in which a BTK inhibitor or a pharmaceutically acceptable salt thereof is administered to a subject weekly; and the discontinuance period is about two weeks.

For intravitreal or intraocular injection to the eye, the first administration period, the second administration period, and the discontinuance period are independently selected from the group consisting of one month, two months, three months, four months, five months, six months, seven months, eight months, nice months, ten months, eleven months, and a year, in which the pharmaceutical composition comprising a BTK inhibitor or a pharmaceutically acceptable salt thereof is administered to a subject monthly, bi-monthly, once every three-month, once every four-month, once every five-month, once every six-month, or yearly. In an embodiment, the first administration period is the same length as the second administration period. In an embodiment, the first administration period is shorter than the second administration period. In an embodiment, the first administration period is longer than the second administration period.

1. A method of treating an ophthalmic condition in a human subject in need thereof comprising: administering to the human subject an amount of a Bruton's Tyrosine Kinase (BTK) inhibitor compound effective to treat the ophthalmic condition in the human subject.

2. The method of claim 1, wherein the BTK inhibitor compound is 1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof.

3. The method of claim 1, wherein administering the BTK inhibitor compound reduces inflammation in an eye of the human subject.

4. The method of claim 2, wherein administering the BTK inhibitor compound reduces inflammation in an eye of the human subject.

5. The method of any one of claims 1 to 4, wherein the ophthalmic condition is ocular inflammation.

6. The method of any one of claims 1 to 4, wherein the ophthalmic condition is selected from dry eye disease, uveitis, post-operative ocular inflammation, corneal transplantation, ocular graft-versus-host disease (GVHD), allergy, allergic conjunctivitis, non-allergic conjunctivitis, or infectious conjunctivitis.

7. The method of any one of claims 1 to 4, wherein the ophthalmic condition is dry eye disease.

8. The method of any one of claims 1 to 4, wherein the ophthalmic condition is aqueous-deficient dry eye disease.

9. The method of any one of claims 1 to 4, wherein the ophthalmic condition is hyperevaporative dry eye disease.

10. The method of any one of claims 1 to 4, wherein the ophthalmic condition is mixed aqueous-deficient and hyperevaporative dry eye disease.

11. The method of any one of claims 1 to 4, wherein the ophthalmic condition is uveitis.

12. The method of any one of claims 1 to 4, wherein the ophthalmic condition is infectious uveitis.

13. The method of any one of claims 1 to 4, wherein the ophthalmic condition is non-infectious uveitis.

14. The method of any one of claims 1 to 4, wherein the ophthalmic condition is anterior uveitis.

15. The method of any one of claims 1 to 4, wherein the ophthalmic condition is intermediate uveitis.

16. The method of any one of claims 1 to 4, wherein the ophthalmic condition is posterior uveitis.

17. The method of any one of claims 1 to 4, wherein the ophthalmic condition is panuveitis.

18. The method of any one of claims 1 to 17, wherein the administering comprises topical administration to an eye of the human subject.

19. The method of any one of claims 1 to 17, wherein the administering comprises intraocular injection to an eye of the human subject.

20. The method of any one of claims 1 to 17, wherein the administering comprises intravitreal injection to an eye of the human subject.

21. The method of any one of claims 1 to 17, wherein the administering comprises periocular administration to the human subject.

22. The method of any one of claims 1 to 17, wherein the administering comprises oral administration to the human subject.

23. The method of any one of claims 1 to 17, wherein the administering comprises intravenous injection to the human subject.

24. The method of any one of the preceding claims, wherein the compound is administered as nanoparticles comprising the compound.

25. The method of any one of the preceding claims, wherein the compound is in a dosage form selected from a solution, suspension, emulsion, microemulsion, ointment, gel, hydrogel, drug delivery device, tablet, or capsule.

26. The method of claim 25, wherein the drug delivery device is an ocular insert for sustained release of the BTK inhibitor compound.

27. The method of claim 25, wherein the dosage form is a sustained release form, an extended release form, a controlled release form, or a combination thereof.

28. The method of claim 27, wherein the sustained release, extended release, or controlled release dosage form comprises a pegylated BTK inhibitor.

29. The method of any one of the preceding claims, wherein the compound is administered as particles that self-aggregate into a depot upon administration.

30. The method of claim 29, wherein the particles further comprise a polymer.

31. The method of claim 30, wherein the polymer is selected from the group consisting of chitosan, gelatin, sodium alginate, albumin, poly-L-lactide (PLLA), poly(lactic acid) (PLA), poly(glycolic acid) (PGA), poly(lactic co-glycolic acid) (PLGA), polycaprolactone, poly(lactide co-caprolactone), poly(methyl methacrylates), poloxamer, poly(ethylene glycol) (PEG), PEG-PLLA, PEG-PLGA, poly(methyl vinyl ether/maleic anhydride), cellulose acetate phthalate, and combinations thereof.

32. The method of claim 30, wherein the polymer is poly(lactic co-glycolic acid) (PLGA), PEG-PLGA, or a combination thereof.

33. The method of any one of the preceding claims, wherein T-cells in an eye of the human subject overexpress lymphocyte function-associated antigen (LFA-1).

34. The method of claim 33, wherein administering the compound decreases the expression of LFA-1.

35. The method of any one of the preceding claims, wherein the compound inhibits intercellular adhesion molecule 1 (ICAM-1) in an eye of the human subject.

36. The method of claim 35, wherein the ICAM-1 is present on antigen-presenting cells (APCs) in the eye of the human subject.

37. The method of claim 35, wherein the ICAM-1 is present on vascular endothelial cells in the eye of the human subject.

38. The method of claim 35, wherein the ICAM-1 is present on corneal epithelial cells in the eye of the human subject.

39. The method of any one of the preceding claims, wherein administering the compound reduces levels of inflammatory cytokines.

40. The method of claim 39, wherein the inflammatory cytokines are selected from IL-1β, IL-6, INF-γ, TNF-α, or a combination thereof.

41. The method of any one of the preceding claims, wherein administering the compound reduces ocular surface APCs, maturation of APCs, or both.

42. The method of claim 41, wherein the APCs are monocytes, macrophages, dendritic cells, B cells, or combinations thereof.

43. The method of any one of the preceding claims, wherein the human subject has a marker of an ophthalmic condition.

44. The method of claim 43, wherein the marker is elevated inflammatory cytokines, elevated chemokines, elevated matrix metalloproteinases (MMPs), elevated toll-like receptor 2 (TLR2), elevated nuclear factor-kappa B (NF-κB), elevated tumor necrosis factor alpha (TNF-α), or combinations thereof.

45. The method of claim 44, wherein the inflammatory cytokines are selected from IL-1β, IL-6, INF-γ, TNF-α, or a combination thereof.

46. The method of any one of the preceding claims, wherein the human subject has an auto-immune disease or an inflammatory disease in addition to the ophthalmic condition.

47. The method of claim 46, wherein the auto-immune disease or inflammatory disease is rheumatoid arthritis, Sjögren's syndrome, Vogt-Koyanagi-Harada (VKH) disease, juvenile idiopathic arthritis, Behçet's disease, systemic sarcoidosis, spondyloarthropathy (such as HLA-B27 associated spondyloarthropathy), Blau syndrome, or IgG-4 related disease (IgG4-RD).

48. The method of any one of the preceding claims, wherein administering occurs at a frequency of three times a daily, twice daily, once daily, every other day, three times a week, twice a week, weekly, every two weeks, twice a month, monthly, every two months, or every three months.

49. A method of reducing an immune response in a human subject having an ophthalmic condition, comprising administering to the human subject an amount of a Bruton's Tyrosine Kinase (BTK) inhibitor compound effective to reduce an immune response in the human subject.

50. The method of claim 49, wherein the immune response is an innate immune response, an adaptive immune response, or both.
